SEQUENCE LISTING (2 sequences)

SEQ ID NO: 1 - protein, 14 aa, synthetic construct
  REGION 1..14: note = Substrate Peptide
KTSAVLQSGFRKME

SEQ ID NO: 2 - protein, 7 aa, synthetic construct
  REGION 1..7: note = Internal Standard Peptide
KTSAVLQ

(19) United States
(12) Patent Application Publication
(10) Pub. No.: US 2025/0025459 A1
(43) Pub. Date: Jan. 23, 2025

(54) COVID-19 TREATMENT MEDICINE CHARACTERIZED BY COMBINING 3CL PROTEASE INHIBITOR AND COVID-19 TREATMENT DRUG

(71) Applicant: Shionogi & Co., Ltd., Osaka (JP)

(72) Inventors: Ryuichi YANO, Osaka-shi, Osaka (JP); Atsuko YAMAMOTO, Osaka-shi, Osaka (JP); Haruaki NOBORI, Osaka-shi, Osaka (JP); Teruhisa KATO, Osaka-shi, Osaka (JP)

(73) Assignee: Shionogi & Co., Ltd., Osaka (JP)

(21) Appl. No.: 18/711,747

(22) PCT Filed: Nov. 25, 2022
(86) PCT No.: PCT/JP2022/043490

§ 371 Date: May 20, 2024

(30) Foreign Application Priority Data
Nov. 26, 2021 (JP) 2021-191636

(51) Int. Cl. A61K 31/495; A61P 31/14; C07K 16/10

(52) U.S. Cl. CPC A61K 31/495; A61P 31/14; C07K 16/1003

(57) ABSTRACT

The present invention provides a medicament useful in treatment and/or prevention, etc. of COVID-19.

Provided is a medicament characterized by combining (A) a compound represented by Formula (I):

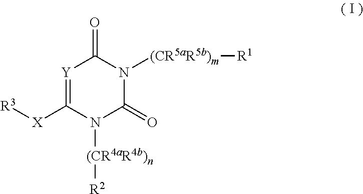

wherein Y is N; R¹ is substituted or unsubstituted aromatic heterocyclyl; R² is substituted or unsubstituted 6-membered aromatic carbocyclyl; R³ is substituted or unsubstituted aromatic heterocyclyl; —X— is —NH—; m is 0 or 1; R^{5a} is a hydrogen atom; R^{5b} is a hydrogen atom; n is 1; R^{4a} is a hydrogen atom; and R^{4b} is a hydrogen atom, or a pharmaceutically acceptable salt thereof; and
(B) a COVID-19 exacerbation suppressant.

### TECHNICAL FIELD

The present invention relates to a medicament characterized by comprising a specific combination of a therapeutic and/or prophylactic agent for coronavirus disease 2019 (COVID-19).

### BACKGROUND ART

Coronaviruses belonging to the subfamily Orthocoronavirinae in the family Coronaviridae, order Nidovirales have a genome size of approximately 30 kilobases and are the largest single-stranded plus-stranded RNA viruses in known RNA viruses. Coronaviruses are classified into four genera of Alphacoronavirus, Betacoronavirus, Gammacoronavirus, and Deltacoronavirus, and seven kinds in total of two kinds of the genus Alphacoronavirus (HCoV-229E and HCoV-NL63) and five kinds of the genus Betacoronavirus (HCoV-HKU1, HCoV-OC43, SARS-CoV, MERS-CoV, and SARS-CoV-2) are known as coronaviruses that infect humans. Among them, four kinds (HCoV-229E, HCoV-NL63, HCoV-HKU1, and HCoV-OC43) are pathogens of cold, and the remaining three kinds are severe acute respiratory syndrome (SARS) coronavirus (SARS-CoV), Middle East respiratory syndrome (MERS) coronavirus (MERS-CoV), and novel coronavirus (SARS-CoV-2) that cause severe pneumonia.

Coronavirus disease 2019 (COVID-19) outbreak in Wuhan, China in December 2019 has internationally widespread rapidly, and WHO announced pandemic on Mar. 11, 2020. The number of infected persons confirmed on Sep. 21, 2022 reaches 650 million or more, and the number of deaths reaches 6.1 million or more (Non-patent Document 1). Contact infection and aerosol infection have been reported as the main infection route of SARS-CoV-2, and it has been confirmed that SARS-CoV-2 keeps drifting in air with aerosols for about 3 hours and maintains infectivity (Non-patent Document 2). The incubation period is about 2 to 14 days, and cold-like symptoms such as fever (87.9%), dry coughing (67.7%), malaise (38.1%), and phlegm (33.4%) are typical (Non-patent Document 3). In severe cases, respiratory failures caused by acute respiratory distress syndrome, acute pulmonary disorder, interstitial pneumonia, etc. occur. Furthermore, multiple organ failures such as renal failure and hepatic failure have also been reported.

In Japan, as a result of drug repositioning of existing drugs, remdesivir, which is an antiviral drug, dexamethasone, which is an anti-inflammatory drug, and baricitinib, which is an antirheumatic drug, have been approved as therapeutic agents against COVID-19, and in January 2022, tocilizumab, which is an anti-IL-6 receptor antibody, have been received additional approval. Furthermore, Ronapreve (Casirivimab and Imdevimab), which is an antibody cocktail therapy (combination administration of an anti-SARS-CoV-2 monoclonal antibody) has been exceptionally approved in July 2021, XEVUDY (Sotrovimab), which is an anti-SARS-CoV-2 monoclonal antibody used as a single agent, has been exceptionally approved in September 2021, and Molnupiravir has been exceptionally approved in December 2021. Sufficient evidences have not been obtained for efficacy and safety of these medicaments, and emergence of resistant strains. Therefore, creating a therapeutic agent for COVID-19 is urgent.

Currently, a method of using therapeutic agents for COVID-19 in combination has been studied for the purpose of reducing the tolerance of SARS-CoV-2, enhancing the therapeutic effect and/or reducing side effects, etc. However, the number of medicaments used for combination is limited, and satisfactory effects are not necessarily obtained.

Research on compounds and antibodies acting on various mechanisms that have an effect on SARS-CoV-2 as a candidate for a therapeutic agent for COVID-19 has been conducted. For example, an anti-SARS-CoV-2 monoclonal antibody, an RNA-dependent RNA polymerase inhibitor, a 3CL protease inhibitor, a TMPRSS2 inhibitor, and the like are exemplified.

Upon infection of cells, coronaviruses synthesize various proteins required for self-replication. There are two polyproteins in the proteins, replication complexes producing viral genomes, and two proteases are included. Proteases cleave polyproteins synthesized from viruses and act indispensably to cause each protein to function. Of two proteases, a protease mostly taking charge of cleaving polyproteins is a 3CL protease (main protease) (Non-patent Document 4).

Regarding COVID-19 therapeutic agents targeting 3CL proteases, it was published in ClinicalTrials.gov in June 2021 that Phase 1b trials for Lufotrelvir (PF-07304814), which is a prodrug of PF-00835231, have completed by Pfizer Inc (NCT04535167). Furthermore, Pfizer Inc. announced in March 2021 to start Phase 1 test of the therapeutic agent PF-07321332 for coronavirus disease 2019. The structural formulae of PF-00835231, Lufotrelvir and PF-07321332 are as shown below, and these agents are different from the compound represented by (A) in the medicament of the present invention in chemical structure (Non-patent Documents 5, 12 and 13 and Patent Documents 5 and 6).

### PF-00835231:

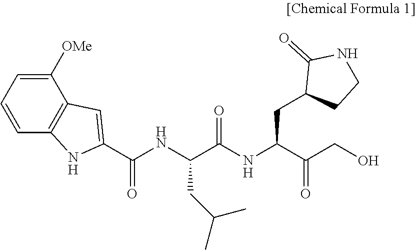 [SMILES: COc1cccc2[nH]c(C(=O)N[C@@H](CC(C)C)C(=O)N[C@@H](C[C@@H]3CCNC3=O)C(=O)CO)cc12]

### Lufotrelvir (PF-07304814):

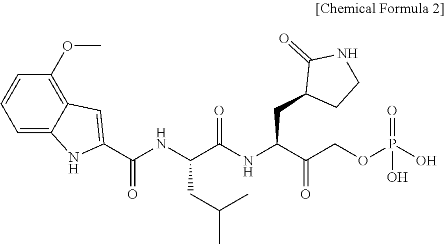 [SMILES: COc1cccc2[nH]c(C(=O)N[C@@H](CC(C)C)C(=O)N[C@@H](C[C@@H]3CCNC3=O)C(=O)COP(=O)(O)O)cc12]

### PF-07321332:

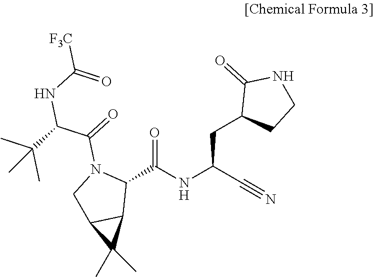 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C]

Further, it has been posted on ClinicalTrials.gov in July 2021 that Phase 2/3 test of combination use of PF-07321332 and Ritonavir for COVID-19 patients having high risk factors was started (NCT04960202). Ritonavir acts as a pharmacokinetic booster by inhibiting the CYP3A-mediated metabolism of a medicament. Moreover, in November 2021, it was reported on the Pfizer website, PAXLOVID™ (PF-07321332; ritonavir) reduced the risk of hospitalization or death by 89% in high-risk adult patients compared to placebo (Non-patent Document 14). Furthermore, in December 2021, PAXLOVID™ was approved for emergency use in the United States, and on Feb. 10, 2022, the Paxlovid (registered trademark) PACK was exceptionally approved in Japan.

In the evaluation of the antiviral effect in vitro, an additive synergistic effect was observed by combining PF-07321332 with Molnupiravir or interferon beta (Non-patent Documents 15 and 16).

Although compounds having 3CL protease inhibitory activity are disclosed in Non-patent Documents 5 to 8, a method of treating COVID-19 by combining the compound represented by (A) in the medicament of the present invention and a compound having 3CL protease inhibitory activity with another COVID-19 therapeutic agent has neither been described nor suggested in any literatures.

Although triazine derivatives having P2X₃ and/or P2X_{2/3} receptor antagonistic activity are disclosed in Patent Documents 1 to 4, the medicament comprising the compound having 3CL protease inhibitory activity and antiviral effect has neither been described nor suggested in any literatures.

Although triazine derivatives having antitumor effects are disclosed in Non-patent Documents 9 to 11, coronavirus 3CL protease inhibitory activity and antiviral effect have neither been described nor suggested.

### PRIOR ART REFERENCES

### Patent Document

[Patent Document 1] International Publication WO 2012/020749 A
[Patent Document 2] International Publication WO 2013/089212 A
[Patent Document 3] International Publication WO 2010/092966 A
[Patent Document 4] International Publication WO 2014/200078 A
[Patent Document 5] International Publication WO 2021/205298 A
[Patent Document 6] International Publication WO 2021/250648 A

### Non-Patent Document

[Non-patent Document 1] “COVID-19 Dashboard by the Center for Systems Science and Engineering at Johns Hopkins University”, [online], Johns Hopkins University, [searched on Sep. 21, 2022], Internet <URL: https://coronavirus.jhu.edu/map.html>
[Non-patent Document 2] The NEW ENGLAND JOURNAL of MEDICINE (2020), Vol. 382, pp. 1564 to 1567
[Non-patent Document 3] “Report of the WHO-China Joint Mission on Coronavirus Disease 2019 (COVID-19)”, [online], Feb. 28, 2020, WHO, [searched on Sep. 21, 2022], Internet <URL: https://www.who.int/docs/default-source/coronaviruse/who-china-joint-mission-on-covid-19-final-report.pdf>
[Non-patent Document 4] Science (2003), Vol. 300, pp. 1763 to 1767
[Non-patent Document 5] “A comparative analysis of SARS-CoV-2 antivirals characterizes 3CLpro inhibitor PF-00835231 as a potential new treatment for COVID-19”, Journal of Virology, [online], Feb. 23, 2021, [searched on Sep. 21, 2022], Internet <URL: https://jvi.asm.org/content/early/2021/02/19/JVI.01819-20><doi: 10.1128/JVI.01819-20>
[Non-patent Document 6] Cell Research (2020), Vol. 30, pp. 678 to 692
[Non-patent Document 7] Science (2020), Vol. 368, pp. 409 to 412
[Non-patent Document 8] ACS Central Science (2021), Vol. 7, No. 3, pp. 467 to 475
[Non-patent Document 9] Cancer Treatment Reviews (1984), Vol. 11, Supplement 1, pp. 99 to 110
[Non-patent Document 10] Contributions to Oncology (1984), Vol. 18, pp. 221 to 234
[Non-patent Document 11] Arzneimittel-Forschung (1984), Issue 11, Vol. 6, pp. 663 to 668
[Non-patent Document 12] 261st Am Chem Soc (ACS) Natl Meet—2021 Apr. 5/2021 Apr. 16—Virtual, N/A Abst 243
[Non-patent Document 13] Science (2021), Vol. 374, pp. 1586 to 1593
[Non-patent Document 14] “Pfizer's Novel COVID-19 Oral Antiviral Treatment Candidate Reduced Risk Of Hospitalization Or Death By 89% In Interim Analysis Of Phase 2/3 EPIC-HR Study”, [online], Nov. 5, 2021, Pfizer Press Release, [retrieved on Sep. 21, 2022], Internet <URL: https://www.pfizer.com/news/press-release/press-release-detail/pfizers-novel-covid-19-oral-antiviral-treatment-candidate>
[Non-patent Document 15] Microorganisms 2022, Vol. 10, 1475
[Non-patent Document 16] Journal of Infection 2022, Vol. 85, pp. 573 to 607

### SUMMARY OF THE INVENTION

### Problems to be Solved by the Invention

An object of the present invention is to provide a medicament having high efficacy with respect to COVID-19 by using a compound having coronavirus 3CL protease inhibitory activity in combination with a different medicament, the medicament being useful in treatment and/or prevention, etc. of coronavirus disease 2019. Preferably, the present invention provides a medicament useful in treatment and/or prevention of COVID-19 with less emergence of low sensitive viruses and few side effects.

### Means for Solving the Problem

The present invention relates to the following.

(1) A medicament characterized by combining (A) a compound represented by Formula (I):

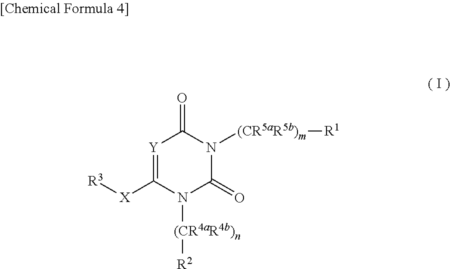

wherein Y is N;
R¹ is substituted or unsubstituted aromatic heterocyclyl;
R² is substituted or unsubstituted 6-membered aromatic carbocyclyl;
R³ is substituted or unsubstituted aromatic heterocyclyl;
—X— is —NH—;
m is 0 or 1;
R^{5a} is a hydrogen atom;
R^{5b} is a hydrogen atom;
n is 1;
R^{4a} is a hydrogen atom; and
R^{4b} is a hydrogen atom, or a pharmaceutically acceptable salt thereof; and
(B) a COVID-19 exacerbation suppressant (provided that, excluding the compound represented by Formula (I) or a pharmaceutically acceptable salt thereof).

(2) The medicament described in the above-described item (1), wherein R¹ is substituted or unsubstituted 5- to 6-membered aromatic heterocyclyl.

(3) The medicament described in the above-described item (1) or (2), wherein
R² is 6-membered aromatic carbocyclyl substituted with one, two, or three substituents selected from a substituent group G;
the substituent group G described here is a group consisting of halogen, cyano, and unsubstituted alkyl.

(4) The medicament described in any one of the above-described items (1) to (3), wherein R³ is substituted or unsubstituted 9- to 10-membered aromatic heterocyclyl.

(5) The medicament described in any one of the above-described items (1) to (4), wherein (A) is a compound represented by formula:

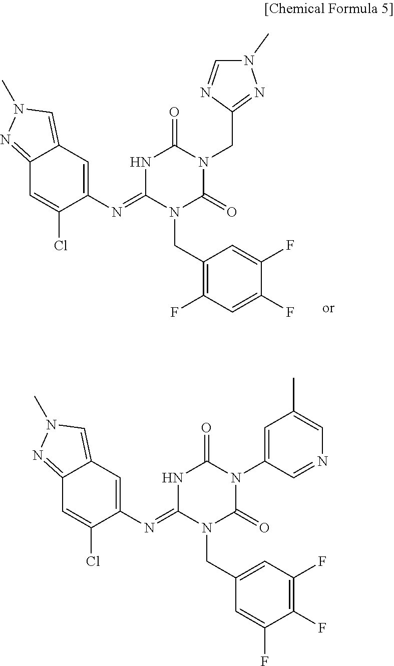 [SMILES: Cn1cnc(Cn2c(=O)[nH]/c(=N\c3cc4cn(C)nc4cc3Cl)n(Cc3cc(F)c(F)cc3F)c2=O)n1 ; Cc1cncc(-n2c(=O)[nH]/c(=N\c3cc4cn(C)nc4cc3Cl)n(Cc3cc(F)c(F)c(F)c3)c2=O)c1]

or a pharmaceutically acceptable salt thereof.

(6) The medicament described in any one of the above-described items (1) to (5), wherein (A) is a compound represented by Formula (I-B):

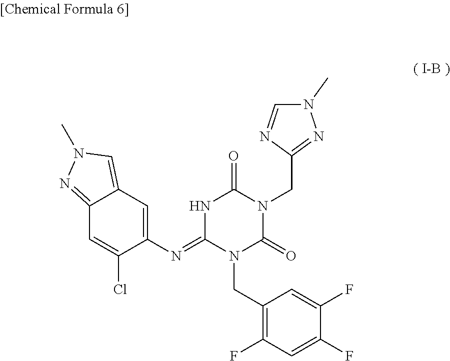 [SMILES: Cn1cnc(Cn2c(=O)[nH]/c(=N\c3cc4cn(C)nc4cc3Cl)n(Cc3cc(F)c(F)cc3F)c2=O)n1]

or a pharmaceutically acceptable salt thereof.

(7) The medicament described in any one of the above-described items (1) to (6), wherein (B) is at least one selected from the group consisting of an anti-SARS-CoV-2 monoclonal antibody, an RNA-dependent RNA polymerase inhibitor, a 3CL protease inhibitor (provided that, excluding the compound represented by Formula (I) or a pharmaceutically acceptable salt thereof), and a TMPRSS2 inhibitor.

(8) The medicament described in any one of the above-described items (1) to (7), wherein (B) is at least one antibody or compound or a pharmaceutically acceptable salt thereof selected from (i) or (ii):
(i) Casirivimab, Imdevimab, Sotrovimab, Tixagevimab, Cilgavimab, or Bebtelovimab,
(ii) Molnupiravir, Remdesivir, AT-527, PF-07321332, PF-00835231, GC376, or Camostat, or a pharmaceutically acceptable salt thereof.

(9) The medicament described in any one of the above-described items (1) to (8), wherein (B) is at least one antibody or compound or a pharmaceutically acceptable salt thereof selected from (i) or (ii):
(i) Casirivimab or Imdevimab,
(ii) Molnupiravir, Remdesivir, PF-07321332, or Camostat, or a pharmaceutically acceptable salt thereof.

(10) The medicament described in any one of the above-described items (1) to (9), wherein (B) is Casirivimab and Imdevimab.

(11) The medicament described in any one of the above-described items (1) to (10), wherein (A) and (B) are administered concomitantly.

(12) The medicament described in any one of the above-described items (1) to (10) which is a combination drug.

(13) The medicament described in any one of the above-described items (1) to (12) which is used for treating and/or preventing coronavirus disease 2019.

(14) The medicament described in any one of the above-described items (1) to (13) which is used for treating and/or preventing infective disease due to SARS-CoV-2.

(15) The medicament described in the above-described item (14), wherein SARS-CoV-2 is a low sensitive virus.

(16) An enhancer for (B) described in the above-described item (1), comprising (A) described in the above-described item (1).

(17) An enhancer for (A) described in the above-described item (1), comprising (B) described in the above-described item (1).

(18) A medicament for administration in combination with (B) described in the above-described item (1), comprising (A) described in the above-described item (1) as an active ingredient.

(19) A medicament for administration in combination with (A) described in the above-described item (1), comprising (B) described in the above-described item (1) as an active ingredient.

(20) The medicament or the enhancer described in any one of the above-described items (1) to (19) which reduces an emergence frequency of a low sensitive virus to a medicament represented as (A).

(21) The medicament or the enhancer described in any one of the above-described items (1) to (20) which is used for an adult aged 50 years or older.

(22) The medicament or the enhancer described in any one of the above-described items (1) to (21) which is used for an adult aged 65 years or older.

(23) The medicament or the enhancer described in any one of the above-described items (1) to (22) which is used for a non-vaccinated patient against SARS-CoV-2.

(24) The medicament or the enhancer described in any one of the above-described items (1) to (23) which is used for an immune-compromised patient.

(25) The medicament or the enhancer described in any one of the above-described items (1) to (24) which is used for a patient that falls into at least one selected from the group consisting of (i) 50 years old or older, (ii) obesity (for example, BMI >30 kg/m² or more), (iii) cardiovascular disease (for example, including hypertension), (iv) asthma or chronic pulmonary disease, (v) Type 1 or 2 diabetes, (vi) chronic renal impairment (for example, including dialyzed patients), (vii) chronic hepatic disease, (viii) immunosuppressed state (for example, malignancy treatment, bone marrow or organ transplantation, immune deficiency, uncontrolled HIV, AIDS, sickle-cell anemia, thalassemia, long-term administration of an immunosuppressant, etc.), (ix) chronic obstructive pulmonary disease (COPD), (x) hyperlipidemia, (xi) smoking, (xii) immune deficiency after solid organ transplantation, (xiii) pregnancy, (xiv) a patient having a neurodevelopmental disease or complicated clinical conditions (for example, cerebral palsy, congenital disease, etc.), and (xv) a patient having a high degree of medical dependence (for example, tracheostomy, gastric fistula, positive pressure ventilation, etc.).

(26) The medicament or the enhancer described in any one of the above-described items (1) to (25) which is used for a patient having pneumonia caused by SARS-CoV-2.

(27) The medicament or the enhancer described in any one of the above-described items (1) to (26) which is used for a patient that falls into at least one selected from the group consisting of (i) a patient with installation of extracorporeal membrane oxygenation (ECMO), (ii) a patient with installation of inhalator, (iii) a patient in ICU, and (iv) a patient having an oxygen saturation (SpO₂) of 93% (room air) or less or requiring oxygen inhalation.

(28) The medicament or the enhancer described in any one of the above-described items (1) to (27) which is used for a patient that falls into at least one selected from the group consisting of (i) an oxygen saturation (SpO₂) of less than 94% (room air, sea level), (ii) PaO₂/FiO₂ of less than 300 mmHg, (iii) a respiration rate of 30 or more/min, and (iv) pulmonary infiltration of 50% or more.

### Effect of the Invention

The compound represented by (A) in the medicament of the present invention has inhibitory activity against the coronavirus 3CL protease, and the medicament of the present invention is useful as a therapeutic agent and/or prophylactic agent for COVID-19 and an enhancer for the COVID-19 exacerbation suppressant.

### BRIEF DESCRIPTION OF DRAWINGS

FIG. 1 shows X-ray powder diffraction patterns of fumaric acid cocrystal Form I (Form I) of a compound represented by Formula (I-B). The horizontal axis represents 2θ (°) and the vertical axis represents intensity (Count).

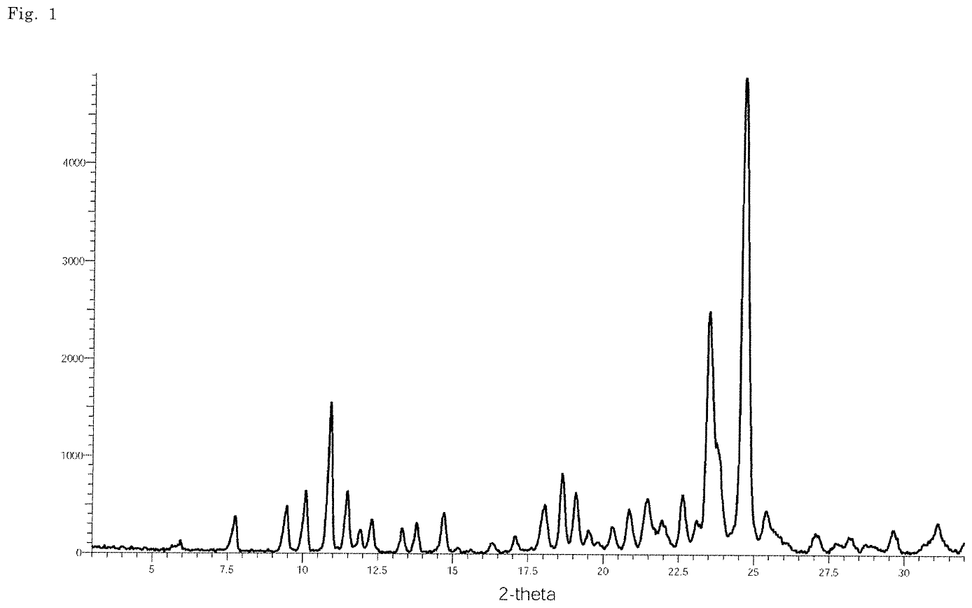

FIG. 2 shows a structure diagram of fumaric acid cocrystal Form I (Form I) of the compound represented by Formula (I-B) in an asymmetric unit.

### MODE FOR CARRYING OUT THE INVENTION

The meanings of the terms as used herein are described below. Unless otherwise specified, each term has the same meaning when used alone or in combination with other terms.

The term “consisting of” means to have only the described elements.

The term “comprising” means not to limit to the described elements and not to exclude undescribed elements.

Hereinafter, the present invention will be described with showing embodiments. It should be understood that, throughout the present specification, the expression of a singular form includes the concept of its plural form unless specified otherwise. Therefore, it should be understood that the article of the singular form (for example, in English, “a”, “an”, “the”, and the like) includes the concept of its plural form unless specified otherwise.

Furthermore, it should be understood that the terms used herein are used in a meaning normally used in the art unless specified otherwise. Thus, unless defined otherwise, all technical and scientific terms used herein have the same meaning as those generally understood by those skilled in the art in the field to which the present invention pertains. If there is a contradiction, the present specification (including definitions) precedes.

Examples of the “COVID-19 exacerbation suppressant” of (B) to be combined with (A) include an anti-SARS-CoV-2 agent, an immunomodulator, and an immunosuppressant. However, the “COVID-19 exacerbation suppressant” used as (B) is a compound or antibody that is different from the compound represented by Formula (I) or a pharmaceutically acceptable salt thereof that is used as (A). Furthermore, the “COVID-19 exacerbation suppressant” used as (B) may be one or two or more medicaments and is not limited to one agent.

The “COVID-19 exacerbation suppressant” is not limited to ones that are commercially available or under development, but examples of ones that are commercially available or under development include Casirivimab, Imdevimab, Sotrovimab, Tixagevimab, Cilgavimab, Bebtelovimab, Molnupiravir, Remdesivir, AT-527, PF-07321332, PF-00835231, and Camostat. Particularly, Casirivimab, Imdevimab, Sotrovimab, Tixagevimab, Cilgavimab, Bebtelovimab, Molnupiravir, Remdesivir, PF-07321332, and Camostat are preferred. Further, Casirivimab and Imdevimab are preferred.

Examples of the “COVID-19 exacerbation suppressant” include mixtures of two neutralizing antibody drugs, known as antibody cocktail therapy, and examples thereof include Casirivimab and Imdevimab, and Tixagevimab and Cilgavimab.

As the anti-SARS-CoV-2 agent, a compound or antibody of which the EC₅₀ value as measured according to the method described in Test Example 1 is less than 100 μM, preferably less than 100 nM can be used.

Examples of the anti-SARS-CoV-2 agent include an anti-SARS-CoV-2 monoclonal antibody, an anti-SARS-CoV-2 polyclonal antibody, an RNA-dependent RNA polymerase inhibitor, a 3CL protease inhibitor (provided that, excluding the compound represented by the above Formula (I) or a pharmaceutically acceptable salt thereof), and a TMPRSS2 inhibitor.

Examples of the “anti-SARS-CoV-2 monoclonal antibody” include Casirivimab (REGN10933), Imdevimab (REGN10987), Bamlanivimab (LY-CoV555), Etesevimab (LY-CoVO16), Sotrovimab (VIR-7831, GSK4182136), AZD7442 (Tixagevimab: AZD8895 and Cilgavimab: AZD1061), Regdanvimab (CT-P59), TY-027, BRII-196, BRII-198, Bebtelovimab (LY-CoV1404), STI-2020, BI-767551 (DZIF-10c), VIR-7832, STI-1499, but it is not limited thereto.

Preferred examples of the “anti-SARS-CoV-2 monoclonal antibody” include Casirivimab, Imdevimab, Sotrovimab, Tixagevimab, Cilgavimab, and Bebtelovimab.

Examples of the “anti-SARS-CoV-2 polyclonal antibody” include Sab-185, but it is not limited thereto.

The “RNA-dependent RNA polymerase inhibitor” may be any compound as long as it has RNA-dependent RNA polymerase inhibitory activity and corresponds to the above-described anti-SARS-CoV-2 agent. Furthermore, it may be prodrug forms thereof.

Examples of the “RNA-dependent RNA polymerase inhibitor” include EIDD-1931 (NHC), Molnupiravir (MK-4482, EIDD-2081) that is a prodrug of EIDD-1931, Remdesivir, Favipiravir, AT-511, AT-527 that is a prodrug of AT-511, Enisamium, and Ribavirin, but it is not limited thereto.

Preferred examples of the “RNA-dependent RNA polymerase inhibitor” include Molnupiravir and Remdesivir.

The “3CL protease inhibitor” may be any compound as long as it has 3CL protease inhibitory action and corresponds to the above-described anti-SARS-CoV-2 agent (provided that, excluding the compound represented by the above Formula (I) or a pharmaceutically acceptable salt thereof). Furthermore, it may be prodrug forms thereof.

Examples of the “3CL protease inhibitor” include PF-07321332, PF-00835231, Lopinavir, Darunavir, GC376, EDP-235, and PBI-0451, but it is not limited thereto.

Preferred examples of the “3CL protease inhibitor” include PF-07321332.

The “TMPRSS2 inhibitor” may be any compound as long as it has TMPRSS2 inhibitory action and corresponds to the above-described anti-SARS-CoV-2 agent. Furthermore, it may be prodrug forms thereof.

Examples of the “TMPRSS2 inhibitor” include Camostat, but it is not limited thereto.

Examples of other anti-SARS-CoV-2 agents include interferon beta-la, peginterferon alfa-2b, Peginterferon lambda, 2-deoxy-D-glucose, iota-carrageenan, Nitazoxanide, niclosamide, ensovibep (MP0420), and Pyronaridine-Artesunate, but it is not limited thereto.

Examples of the immunomodulator or the immunosuppressant include a steroid drug, a Janus kinase (JAK) inhibitor, an anti-IL-6 receptor monoclonal antibody, an anti-human IL-16 monoclonal antibody, an IL-1 inhibitor, an anti-human TNFα monoclonal antibody, an anti-CD73 monoclonal antibody, an anti-CCR5 receptor monoclonal antibody, an anti-LIGHT monoclonal antibody, an anti-GM-CSF monoclonal antibody, an anti-GM-CSF receptor monoclonal antibody, a DP1 inhibitor, an H₂ receptor antagonist, an androgen receptor antagonist, a GM-CSF formulation, a calpain inhibitor, a gelsolin stimulant, an anti-human plasma kallikrein monoclonal antibody, an anti-C5 monoclonal antibody, and an antirheumatic.

Examples thereof include dexamethasone, hydrocortisone, methylprednisolone, ciclesonide, budesonide, Baricitinib, Tofacitinib, Tocilizumab, Sarilumab, Levilimab, Canakinumab, Anakinra, Infliximab, mupadolimab (CPI-006), Leronlimab, AVTX-002 (CERC-002), Lenzilumab, Gimsilumab, Otilimab (GSK3196165), Mavrilimumab, ADC-7405, ADC-9971, AM-432, AMG-009, AP-768, AZD-5985, AZD-8075, Laropiprant, ONO-4053, ONO-4127Na, 5-5751, AMG-853, AGN-211377, SAR-389644, Vidupiprant, Asapiprant, Famotidine, Proxalutamide, Sargramostim, Bld-2660, a plasma gelsolin formulation, Lanadelumab, ravulizumab, colchicine, bucillamine, ivermectin, Azithromycin, and interferon beta, but it is not limited thereto.

Herein, the expression “(B) is at least one antibody or compound or a pharmaceutically acceptable salt thereof selected from (i) or (ii):
(i) Casirivimab, Imdevimab, Sotrovimab, Tixagevimab, Cilgavimab, or Bebtelovimab,
(ii) Molnupiravir, Remdesivir, AT-527, PF-07321332, PF-00835231, GC376, or Camostat, or a pharmaceutically acceptable salt thereof” includes all embodiments of an embodiment wherein (B) is at least one antibody selected from (i), an embodiment wherein (B) is at least one compound selected from (ii), or a pharmaceutically acceptable salt thereof, and an embodiment wherein (B) is at least one antibody selected from (i) and at least one compound selected from (ii), or a pharmaceutically acceptable salt thereof.

As an embodiment, examples of (B) include Casirivimab, Imdevimab, and Sotrovimab.

As an embodiment, examples of (B) include Casirivimab and Imdevimab.

As an embodiment, examples of (B) include Imdevimab and Sotrovimab.

As an embodiment, examples of (B) include Casirivimab and Sotrovimab.

As an embodiment, examples of (B) include Casirivimab.

As an embodiment, examples of (B) include Imdevimab.

As an embodiment, examples of (B) include Sotrovimab.

As an embodiment, examples of (B) include Tixagevimab, Cilgavimab, and Bebtelovimab.

As an embodiment, examples of (B) include Bebtelovimab.

As an embodiment, examples of (B) include Tixagevimab and Cilgavimab.

As an embodiment, examples of (B) include Tixagevimab.

As an embodiment, examples of (B) include Cilgavimab.

The compound represented by Formula (I) or a pharmaceutically acceptable salt thereof of (A) will be described below.

“Halogen” includes a fluorine atom, a chlorine atom, a bromine atom, and an iodine atom. In particular, halogen is preferably a fluorine atom and a chlorine atom.

“Alkyl” includes a linear or branched hydrocarbon group having 1 to 15 carbon atoms, preferably 1 to 10 carbon atoms, more preferably 1 to 6 carbon atoms, and further preferably 1 to 4 carbon atoms. Examples thereof include methyl, ethyl, n-propyl, isopropyl, n-butyl, isobutyl, sec-butyl, tert-butyl, n-pentyl, isopentyl, neopentyl, n-hexyl, isohexyl, n-heptyl, isoheptyl, n-octyl, isooctyl, n-nonyl, and n-decyl.

Examples of a preferred embodiment of “alkyl” include methyl, ethyl, n-propyl, isopropyl, n-butyl, isobutyl, sec-butyl, tert-butyl, and n-pentyl. Examples of a further preferred embodiment thereof include methyl, ethyl, n-propyl, isopropyl, and tert-butyl.

“Alkenyl” includes a linear or branched hydrocarbon group having one or more double bond(s) at any position(s) which has 2 to 15 carbon atoms, preferably 2 to 10 carbon atoms, more preferably 2 to 6 carbon atoms, and further preferably 2 to 4 carbon atoms. Examples thereof include vinyl, allyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl, undecenyl, dodecenyl, tridecenyl, tetradecenyl, and pentadecenyl.

Examples of a preferred embodiment of “alkenyl” include vinyl, allyl, propenyl, isopropenyl, and butenyl. Examples of a further preferred embodiment include ethenyl and n-propenyl.

“Alkynyl” includes a linear or branched hydrocarbon group having one or more triple bond(s) at any position(s) which has 2 to 10 carbon atoms, preferably 2 to 8 carbon atoms, more preferably 2 to 6 carbon atoms, and further preferably 2 to 4 carbon atoms. Further, “alkynyl” may have double bond(s) at any position(s). For example, “alkynyl” includes ethynyl, propynyl, butynyl, pentynyl, hexynyl, heptynyl, octynyl, nonynyl, decynyl, and the like.

Examples of a preferred embodiment of “alkynyl” include ethynyl, propynyl, butynyl, and pentynyl. Examples of a further preferred embodiment thereof include ethynyl and propynyl.

“Aromatic carbocyclyl” means a cyclic aromatic hydrocarbon group which is monocyclic or polycyclic having two or more rings. Examples thereof include phenyl, naphthyl, anthryl, and phenanthryl.

Examples of a preferred embodiment of the “aromatic carbocyclyl” include phenyl.

“6-Membered aromatic carbocyclyl” means a cyclic aromatic hydrocarbon group which is monocyclic. Examples thereof include phenyl.

“Non-aromatic carbocyclyl” means a cyclic saturated hydrocarbon group or a cyclic unsaturated non-aromatic hydrocarbon group which is monocyclic or polycyclic having two or more rings. The “non-aromatic carbocyclyl” which is polycyclic having two or more rings also includes a fused ring group wherein a non-aromatic carbocyclyl, which is monocyclic or polycyclic having two or more rings, is fused with a ring of the above-described “aromatic carbocyclyl”.

Further, the “non-aromatic carbocyclyl” also includes a group having a bridge or a group to form a spiro ring as follows.

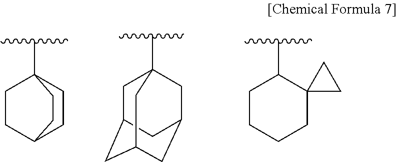 [SMILES: *C12CCC(CC1)CC2 ; *C12CC3CC(CC(C3)C1)C2 ; *C1CCCCC12CC2]

The non-aromatic carbocyclyl which is monocyclic is a carbocyclyl having preferably 3 to 16 carbon atoms, more preferably 3 to 12 carbon atoms, and further preferably 4 to 8 carbon atoms. Examples thereof include cyclopropyl, cyclobutyl, cyclopentyl, cyclohexyl, cycloheptyl, cyclooctyl, cyclononyl, cyclodecyl, cyclopropenyl, cyclobutenyl, cyclopentenyl, cyclohexenyl, cycloheptenyl, and cyclohexadienyl.

The non-aromatic carbocyclyl which is polycyclic having two or more rings is a carbocyclyl having preferably 8 to 20 carbon atoms and more preferably 8 to 16 carbon atoms. Examples thereof include indanyl, indenyl, acenaphthyl, tetrahydronaphthyl, and fluorenyl.

“Aromatic heterocyclyl” means an aromatic cyclic group, which is monocyclic or polycyclic having two or more rings, having one or more, same or different heteroatom(s) selected optionally from O, S, and N.

The aromatic heterocyclyl which is polycyclic having two or more rings include a fused ring group wherein an aromatic heterocyclyl, which is monocyclic or polycyclic having two or more rings, is fused with a ring of the above-described “aromatic carbocyclyl” and may have the binding group at any ring(s).

The aromatic heterocyclyl which is monocyclic is preferably a 5- to 8-membered ring and more preferably a 5- or 6-membered ring. Examples of the 5-membered aromatic heterocyclyl include pyrrolyl, imidazolyl, pyrazolyl, triazolyl, tetrazolyl, furyl, thienyl, isoxazolyl, oxazolyl, oxadiazolyl, isothiazolyl, thiazolyl, and thiadiazolyl. Examples of the 6-membered aromatic heterocyclyl include pyridyl, pyridazinyl, pyrimidinyl, pyrazinyl, and triazinyl.

The aromatic heterocyclyl which is bicyclic is preferably an 8- to 10-membered ring and more preferably a 9- or 10-membered ring. Examples thereof include indolyl, isoindolyl, indazolyl, indolizinyl, quinolinyl, isoquinolinyl, cinnolinyl, phthalazinyl, quinazolinyl, naphthyridinyl, quinoxalinyl, purinyl, pteridinyl, benzimidazolyl, benzisoxazolyl, benzoxazolyl, benzoxadiazolyl, benzisothiazolyl, benzothiazolyl, benzothiadiazolyl, benzofuryl, isobenzofuryl, benzothienyl, benzotriazolyl, imidazopyridyl, triazolopyridyl, imidazothiazolyl, pyrazinopyridazinyl, oxazolopyridyl, and thiazolopyridyl. Examples of the 9-membered aromatic heterocyclyl include indolyl, isoindolyl, indazolyl, indolizinyl, purinyl, benzimidazolyl, benzisoxazolyl, benzoxazolyl, benzoxadiazolyl, benzisothiazolyl, benzothiazolyl, benzothiadiazolyl, benzotriazolyl, benzofuranyl, imidazopyridyl, triazolopyridyl, oxazolopyridyl, and thiazolopyridyl. Examples of the 10-membered aromatic heterocyclyl include quinolinyl, isoquinolinyl, cinnolinyl, phthalazinyl, quinazolinyl, naphthyridinyl, quinoxalinyl, pteridinyl, and pyrazinopyridazinyl.

The aromatic heterocyclyl which is polycyclic having three or more rings is preferably a 13- to 15-membered ring. Examples thereof include carbazolyl, acridinyl, xanthenyl, phenothiazinyl, phenoxathiinyl, phenoxazinyl, and dibenzofuryl.

The “5- to 6-membered aromatic heterocyclyl” means a 5- or 6-membered aromatic heterocyclyl of the above-described “aromatic heterocyclyl”.

The “9- to 10-membered aromatic heterocyclyl” means a 9- or 10-membered aromatic heterocyclyl of the above-described “aromatic heterocyclyl”.

“Non-aromatic heterocyclyl” means a non-aromatic cyclic group, which is monocyclic or polycyclic having two or more rings, having one or more, same or different heteroatom(s) selected optionally from O, S, and N. The non-aromatic heterocyclyl which is polycyclic having two or more rings also includes a fused ring group wherein a non-aromatic heterocyclyl, which is monocyclic or polycyclic having two or more rings, is fused with a ring of each of the above-described “aromatic carbocyclyl”, “non-aromatic carbocyclyl”, and/or “aromatic heterocyclyl” and further, a fused ring group wherein a non-aromatic carbocyclyl, which is monocyclic or polycyclic having two or more rings, is fused with a ring of the above-described “aromatic heterocyclyl”, and may have the binding group at any ring(s).

Further, the “non-aromatic heterocyclyl” also includes a group having a bridge or a group to form a spiro ring as follows.

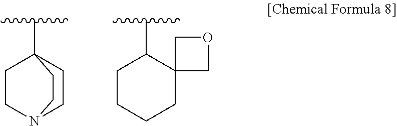 [SMILES: *C12CCN(CC1)CC2 ; *C1CCCCC12COC2]

The non-aromatic heterocyclyl which is monocyclic is preferably a 3- to 8-membered ring and more preferably a 5- or 6-membered ring.

Examples of the 3-membered non-aromatic heterocyclyl include thiiranyl, oxiranyl, and aziridinyl. Examples of the 4-membered non-aromatic heterocyclyl include oxetanyl and azetidinyl. Examples of the 5-membered non-aromatic heterocyclyl include oxathiolanyl, thiazolidinyl, pyrrolidinyl, pyrrolinyl, imidazolidinyl, imidazolinyl, pyrazolidinyl, pyrazolinyl, tetrahydrofuryl, dihydrothiazolyl, tetrahydroisothiazolyl, dioxolanyl, dioxolyl, and thiolanyl. Examples of the 6-membered non-aromatic heterocyclyl include dioxanyl, thianyl, piperidyl, piperazinyl, morpholinyl, morpholino, thiomorpholinyl, thiomorpholino, dihydropyridyl, tetrahydropyridyl, tetrahydropyranyl, dihydrooxazinyl, tetrahydropyridazinyl, hexahydropyrimidinyl, dioxazinyl, thiinyl, and thiazinyl. Examples of the 7-membered non-aromatic heterocyclyl include hexahydroazepinyl, tetrahydrodiazepinyl, and oxepanyl.

The non-aromatic heterocyclyl which is polycyclic having two or more rings is preferably an 8- to 20-membered ring, more preferably an 8- to 13-membered ring, and further preferably 8- to 10-membered ring. Examples thereof include indolinyl, isoindolinyl, chromanyl, and isochromanyl.

The expression “may be substituted with a substituent group α” herein means “may be substituted with one or more groups selected from a substituent group α”. The same is true in substituent groups β, γ, and γ′.

Substituent group α: halogen, hydroxy, carboxy, alkyloxy, haloalkyloxy, alkenyloxy, alkynyloxy, sulfanyl, and cyano.

Substituent group β: halogen, hydroxy, carboxy, cyano, alkyl which may be substituted with the substituent group α, alkenyl which may be substituted with the substituent group α, alkynyl which may be substituted with the substituent group α, alkylcarbonyl which may be substituted with the substituent group α, alkenylcarbonyl which may be substituted with the substituent group α, alkynylcarbonyl which may be substituted with the substituent group α, alkylsulfanyl which may be substituted with the substituent group α, alkenylsulfanyl which may be substituted with the substituent group α, alkynylsulfanyl which may be substituted with the substituent group α, alkylsulfinyl which may be substituted with the substituent group α, alkenylsulfinyl which may be substituted with the substituent group α, alkynylsulfinyl which may be substituted with the substituent group α, alkylsulfonyl which may be substituted with the substituent group α, alkenylsulfonyl which may be substituted with the substituent group α, alkynylsulfonyl which may be substituted with the substituent group α,
an aromatic carbocyclyl which may be substituted with the substituent group γ, a non-aromatic carbocyclyl which may be substituted with the substituent group γ′, an aromatic heterocyclyl which may be substituted with the substituent group γ, a non-aromatic heterocyclyl which may be substituted with the substituent group γ′, aromatic carbocyclylalkyl which may be substituted with the substituent group γ, non-aromatic carbocyclylalkyl which may be substituted with the substituent group γ′, aromatic heterocyclylalkyl which may be substituted with the substituent group γ, non-aromatic heterocyclylalkyl which may be substituted with the substituent group γ′, aromatic carbocyclylcarbonyl which may be substituted with the substituent group γ, non-aromatic arbocyclylcarbonyl which may be substituted with the substituent group γ′, aromatic heterocyclylcarbonyl which may be substituted with the substituent group γ, non-aromatic heterocyclylcarbonyl which may be substituted with the substituent group γ′, aromatic carbocyclyloxycarbonyl which may be substituted with the substituent group γ, non-aromatic carbocyclyloxycarbonyl which may be substituted with the substituent group γ′, aromatic heterocyclyloxycarbonyl which may be substituted with the substituent group γ, non-aromatic heterocyclyloxycarbonyl which may be substituted with the substituent group γ′, aromatic carbocyclylsulfanyl which may be substituted with the substituent group γ, non-aromatic carbocyclylsulfanyl which may be substituted with the substituent group γ′, aromatic heterocyclylsulfanyl which may be substituted with the substituent group γ, non-aromatic heterocyclylsulfanyl which may be substituted with the substituent group γ′, aromatic carbocyclylsulfinyl which may be substituted with the substituent group γ, non-aromatic=carbocyclylsulfinyl which may be substituted with the substituent group γ′, aromatic heterocyclylsulfinyl which may be substituted with the substituent group γ, non-aromatic heterocyclylsulfinyl which may be substituted with the substituent group γ′, aromatic carbocyclylsulfonyl which may be substituted with the substituent group γ, non-aromatic carbocyclylsulfonyl which may be substituted with the substituent group γ′, aromatic heterocyclylsulfonyl which may be substituted with the substituent group γ, and non-aromatic heterocyclylsulfonyl which may be substituted with the substituent group γ′.

Substituent group γ: substituent group α, alkyl, haloalkyl, hydroxyalkyl, alkenyl, alkynyl, alkylcarbonyl, haloalkylcarbonyl, alkenylcarbonyl, and alkynylcarbonyl.

Substituent group γ′: substituent group γ and oxo.

Examples of substituents on the ring of “aromatic carbocycle” and “aromatic heterocycle” of a “substituted aromatic carbocyclyl” and a “substituted aromatic heterocyclyl” include the following substituent group B. An atom at any position(s) on the ring may be bonded to one or more group(s) selected from the following substituent group B.

Substituent group B: halogen, hydroxy, carboxy, formyl, formyloxy, sulfanyl, sulfino, sulfo, thioformyl, thiocarboxy, dithiocarboxy, thiocarbamoyl, cyano, nitro, nitroso, azide, hydrazino, ureide, amidino, guanidino, pentafluorothio, trialkylsilyl,
alkyl which may be substituted with the substituent group α, alkenyl which may be substituted with the substituent group α, alkynyl which may be substituted with the substituent group α, alkyloxy which may be substituted with the substituent group α, alkenyloxy which may be substituted with the substituent group α, alkynyloxy which may be substituted with the substituent group α, alkylcarbonyloxy which may be substituted with the substituent group α, alkenylcarbonyloxy which may be substituted with the substituent group α, alkynylcarbonyloxy which may be substituted with the substituent group α, alkylcarbonyl which may be substituted with the substituent group α, alkenylcarbonyl which may be substituted with the substituent group α, alkynylcarbonyl which may be substituted with the substituent group α, alkyloxycarbonyl which may be substituted with the substituent group α, alkenyloxycarbonyl which may be substituted with the substituent group α, alkynyloxycarbonyl which may be substituted with the substituent group α, alkylsulfanyl which may be substituted with the substituent group α, alkenylsulfanyl which may be substituted with the substituent group α, alkynylsulfanyl which may be substituted with the substituent group α, alkylsulfinyl which may be substituted with the substituent group α, alkenylsulfinyl which may be substituted with the substituent group α, alkynylsulfinyl which may be substituted with the substituent group α, alkylsulfonyl which may be substituted with the substituent group α, alkenylsulfonyl which may be substituted with the substituent group α, alkynylsulfonyl which may be substituted with the substituent group α, amino which may be substituted with the substituent group β, imino which may be substituted with the substituent group β, carbamoyl which may be substituted with the substituent group β, sulfamoyl which may be substituted with the substituent group β,
an aromatic carbocyclyl which may be substituted with the substituent group γ, a non-aromatic carbocyclyl which may be substituted with the substituent group γ′, an aromatic heterocyclyl which may be substituted with the substituent group γ, a non-aromatic heterocyclyl which may be substituted with the substituent group γ′, aromatic carbocyclyloxy which may be substituted with the substituent group γ, non-aromatic carbocyclyloxy which may be substituted with the substituent group γ′, aromatic heterocyclyloxy which may be substituted with the substituent group γ, non-aromatic heterocyclyloxy which may be substituted with the substituent group γ′, aromatic carbocyclylcarbonyloxy which may be substituted with the substituent group γ, non-aromatic carbocyclylcarbonyloxy which may be substituted with the substituent group γ′, aromatic heterocyclylcarbonyloxy which may be substituted with the substituent group γ, non-aromatic heterocyclylcarbonyloxy which may be substituted with the substituent group γ′, aromatic carbocyclylcarbonyl which may be substituted with the substituent group γ, non-aromatic carbocyclylcarbonyl which may be substituted with the substituent group γ′, aromatic heterocyclylcarbonyl which may be substituted with the substituent group γ, non-aromatic heterocyclylcarbonyl which may be substituted with the substituent group γ′, aromatic carbocyclyloxycarbonyl which may be substituted with the substituent group γ, non-aromatic carbocyclyloxycarbonyl which may be substituted with the substituent group γ′, aromatic heterocyclyloxycarbonyl which may be substituted with the substituent group γ, non-aromatic heterocyclyloxycarbonyl which may be substituted with the substituent group γ′, aromatic carbocyclylalkyl which may be substituted with the substituent group γ, non-aromatic carbocyclylalkyl which may be substituted with the substituent group γ′, aromatic heterocyclylalkyl which may be substituted with the substituent group γ, non-aromatic heterocyclylalkyl which may be substituted with the substituent group γ′, aromatic carbocyclylalkyloxy which may be substituted with the substituent group γ, non-aromatic carbocyclylalkyloxy which may be substituted with the substituent group γ′, aromatic heterocyclylalkyloxy which may be substituted with the substituent group γ, non-aromatic heterocyclylalkyloxy which may be substituted with the substituent group γ′, aromatic carbocyclylalkyloxycarbonyl which may be substituted with the substituent group γ, non-aromatic carbocyclylalkyloxycarbonyl which may be substituted with the substituent group γ′, aromatic heterocyclylalkyloxycarbonyl which may be substituted with the substituent group γ, non-aromatic heterocyclylalkyloxycarbonyl which may be substituted with the substituent group γ′, aromatic carbocyclylalkyloxyalkyl which may be substituted with the substituent group γ, non-aromatic carbocyclylalkyloxyalkyl which may be substituted with the substituent group γ′, aromatic heterocyclylalkyloxyalkyl which may be substituted with the substituent group γ, non-aromatic heterocyclylalkyloxyalkyl which may be substituted with the substituent group γ′, aromatic carbocyclylsulfanyl which may be substituted with the substituent group γ, non-aromatic carbocyclylsulfanyl which may be substituted with the substituent group γ′, aromatic heterocyclylsulfanyl which may be substituted with the substituent group γ, non-aromatic heterocyclylsulfanyl which may be substituted with the substituent group γ′, aromatic carbocyclylsulfinyl which may be substituted with the substituent group γ, non-aromatic carbocyclylsulfinyl which may be substituted with the substituent group γ′, aromatic heterocyclylsulfinyl which may be substituted with the substituent group γ, non-aromatic heterocyclylsulfinyl which may be substituted with the substituent group γ′, aromatic carbocyclylsulfonyl which may be substituted with the substituent group γ, non-aromatic carbocyclylsulfonyl which may be substituted with the substituent group γ′, aromatic heterocyclylsulfonyl which may be substituted with the substituent group γ, and non-aromatic heterocyclylsulfonyl which may be substituted with the substituent group γ′.

Examples of substituents on the ring of “non-aromatic carbocycle” and “non-aromatic heterocycle” of a “substituted non-aromatic carbocyclyl” and a “substituted non-aromatic heterocyclyl” include the following substituent group C. An atom at any position(s) on the ring may be bonded to one or more group(s) selected from the following substituent group C.

Substituent group C: substituent group B and oxo.

When the “non-aromatic carbocycle” and the “non-aromatic heterocycle” are substituted with “oxo”, it means a ring in which two hydrogen atoms on the carbon atom are substituted as below.

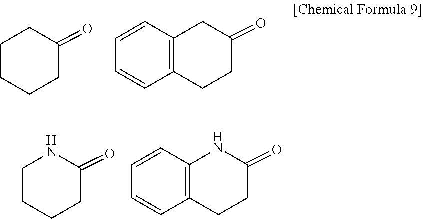

Examples of substituents of the “substituted or unsubstituted aromatic heterocyclyl” or the “substituted or unsubstituted 5- to 6-membered aromatic heterocyclyl” in R¹ include
halogen; and
substituted or unsubstituted alkyl. It may be substituted with one or more group(s) selected from the above substituents.

Examples of substituents of the “substituted or unsubstituted aromatic heterocyclyl” or the “substituted or unsubstituted 5- to 6-membered aromatic heterocyclyl” in R¹ include
halogen;
substituted alkyl (as the substituent, hydroxy); and unsubstituted alkyl. It may be substituted with one or more group(s) selected from the above substituents.

Examples of substituents of the “substituted or unsubstituted 6-membered aromatic carbocyclyl” in R² include
halogen; cyano; and
substituted or unsubstituted alkyl. It may be substituted with one or more group(s) selected from the above substituents.

Examples of substituents of the “substituted or unsubstituted 6-membered aromatic carbocyclyl” in R² include
halogen; cyano;
substituted alkyl (as the substituent, halogen); and unsubstituted alkyl. It may be substituted with one or more group(s) selected from the above substituents.

Examples of substituents of the “substituted or unsubstituted aromatic heterocyclyl” or the “substituted or unsubstituted 9- to 10-membered aromatic heterocyclyl” in R³ include
halogen;
substituted or unsubstituted alkyl; and
a substituted or unsubstituted non-aromatic heterocyclyl. It may be substituted with one or more group(s) selected from the above substituents.

Examples of substituents of the “substituted or unsubstituted aromatic heterocyclyl” or the “substituted or unsubstituted 9- to 10-membered aromatic heterocyclyl” in R³ include
halogen;
substituted alkyl (as the substituent, halogen, hydroxy, alkylcarbonylamino, or a non-aromatic heterocyclyl); unsubstituted alkyl;
substituted non-aromatic heterocyclyl (as the substituent, alkylcarbonyl); and
unsubstituted non-aromatic heterocyclyl. It may be substituted with one or more group(s) selected from the above substituents.

Preferred embodiments of R¹, R², R³, and m in the compound represented by Formula (I):

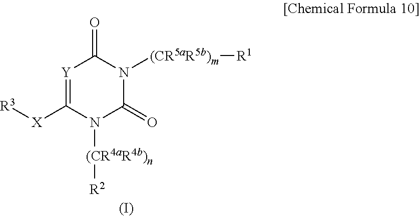

are described below. Examples of the compound represented by Formula (I) include embodiments of all combinations of specific examples described below. Note that, Y, —X—, R^{5a}, R^{5b}, n, R^{4a}, and R^{4b} are as described in the above-described item (1).

As R¹, a substituted or unsubstituted aromatic heterocyclyl is exemplified (hereinafter, referred to as A-1).

As R¹, a substituted or unsubstituted 5- to 6-membered aromatic heterocyclyl is exemplified (hereinafter, referred to as A-2).

As R¹, an aromatic heterocyclyl which is substituted with halogen, substituted alkyl (substituent: hydroxy), or unsubstituted alkyl or an unsubstituted aromatic heterocyclyl is exemplified (hereinafter, referred to as A-3).

As R¹, a 5- to 6-membered aromatic heterocyclyl which is substituted with halogen, substituted alkyl (substituent: hydroxy), or unsubstituted alkyl or an unsubstituted 5- to 6-membered aromatic heterocyclyl is exemplified (hereinafter, referred to as A-4).

As R¹, an aromatic heterocyclyl which is substituted with unsubstituted alkyl or halogen or an unsubstituted aromatic heterocyclyl is exemplified (hereinafter, referred to as A-5).

As R¹, a 5- to 6-membered aromatic heterocyclyl which is substituted with unsubstituted alkyl or halogen or an unsubstituted 5- to 6-membered aromatic heterocyclyl is exemplified (hereinafter, referred to as A-6).

As R¹, an aromatic heterocyclyl which is substituted with unsubstituted alkyl or halogen is exemplified (hereinafter, referred to as A-7).

As R¹, a 5- to 6-membered aromatic heterocyclyl which is substituted with unsubstituted alkyl or halogen is exemplified (hereinafter, referred to as A-8).

As R¹, an aromatic heterocyclyl which is substituted with unsubstituted alkyl or an unsubstituted aromatic heterocyclyl is exemplified (hereinafter, referred to as A-9).

As R¹, a 5- to 6-membered aromatic heterocyclyl which is substituted with unsubstituted alkyl or an unsubstituted 5- to 6-membered aromatic heterocyclyl is exemplified (hereinafter, referred to as A-10).

As R¹, an aromatic heterocyclyl which is substituted with unsubstituted alkyl is exemplified (hereinafter, referred to as A-11).

As R¹, a 5- to 6-membered aromatic heterocyclyl which is substituted with unsubstituted alkyl is exemplified (hereinafter, referred to as A-12).

As R², a substituted or unsubstituted 6-membered aromatic carbocyclyl is exemplified (hereinafter, referred to as B-1).

As R², a 6-membered aromatic carbocyclyl which is substituted with halogen, cyano, substituted alkyl (substituent: halogen) or unsubstituted alkyl is exemplified (hereinafter, referred to as B-2).

As R², a 6-membered aromatic carbocyclyl which is substituted with halogen, cyano, or unsubstituted alkyl is exemplified (hereinafter, referred to as B-3).

As R², a 6-membered aromatic carbocyclyl which is substituted with two to four substituents selected from a substituent group G (substituent group G: halogen, cyano, and unsubstituted alkyl) is exemplified (hereinafter, referred to as B-4).

As R², a 6-membered aromatic carbocyclyl which is substituted with two or three substituents selected from a substituent group G (substituent group G: halogen, cyano, and unsubstituted alkyl) is exemplified (hereinafter, referred to as B-5).

As R², a 6-membered aromatic carbocyclyl which is substituted with three or four substituents selected from a substituent group G (substituent group G: halogen, cyano, and unsubstituted alkyl) is exemplified (hereinafter, referred to as B-6).

As R², a 6-membered aromatic carbocyclyl which is substituted with three halogens is exemplified (hereinafter, referred to as B-7).

As R³, a substituted or unsubstituted aromatic heterocyclyl is exemplified (hereinafter, referred to as C-1).

As R³, a substituted or unsubstituted 9- to 10-membered aromatic heterocyclyl is exemplified (hereinafter, referred to as C-2).

As R³, an aromatic heterocyclyl which is substituted with halogen or substituted or unsubstituted alkyl is exemplified (hereinafter, referred to as C-3).

As R³, a 9- to 10-membered aromatic heterocyclyl which is substituted with halogen or substituted or unsubstituted alkyl is exemplified (hereinafter, referred to as C-4).

As R³, an aromatic heterocyclyl which is substituted with halogen or unsubstituted alkyl is exemplified (hereinafter, referred to as C-5).

As R³, a 9- to 10-membered aromatic heterocyclyl which is substituted with halogen or unsubstituted alkyl is exemplified (hereinafter, referred to as C-6).

As R³, indazolyl which is substituted with halogen or unsubstituted alkyl is exemplified (hereinafter, referred to as C-7).

As R³, indazolyl which is substituted with halogen and unsubstituted alkyl is exemplified (hereinafter, referred to as C-8).

m includes 0 or 1 (hereinafter, referred to as D-1).

m includes 0 (hereinafter, referred to as D-2).

m includes 1 (hereinafter, referred to as D-3).

Examples of the compound represented by Formula (I) include embodiments described below.

(a-1)
R¹ is (A-12);
R² is (B-7);
R³ is (C-8); and
m is (D-2).
   (a-2)
R¹ is (A-12);
R² is (B-7);
R³ is (C-8); and
m is (D-3).
   (a-3)
R¹ is (A-12);
R² is (B-7);
R³ is (C-8); and
m is (D-1).
   (a-4)
R¹ is (A-4);
R² is (B-4);
R³ is (C-4); and
m is (D-1).

The compound represented by Formula (I) is not limited to particular isomers, but includes any possible isomers (for example, keto-enol isomer, imine-enamine isomer, diastereoisomer, optical isomer, rotamer, etc.), racemates, and a mixture thereof. For example, the compound represented by Formula (I) includes a tautomer as shown below.

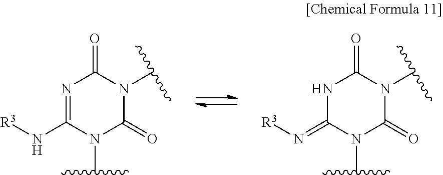

For example, Compound (I-003) includes tautomers as shown below and a mixture thereof.

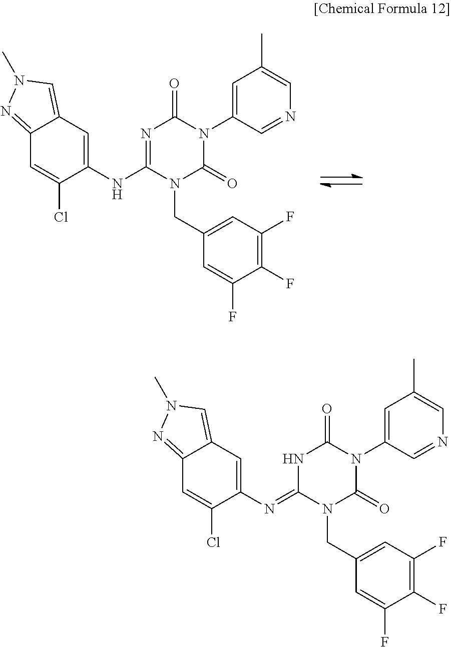 [SMILES: Cc1cncc(-n2c(=O)nc(Nc3cc4cn(C)nc4cc3Cl)n(Cc3cc(F)c(F)c(F)c3)c2=O)c1 ; Cc1cncc(-n2c(=O)[nH]/c(=N\c3cc4cn(C)nc4cc3Cl)n(Cc3cc(F)c(F)c(F)c3)c2=O)c1]

For example, Compound (I-005) includes tautomers as shown below and a mixture thereof.

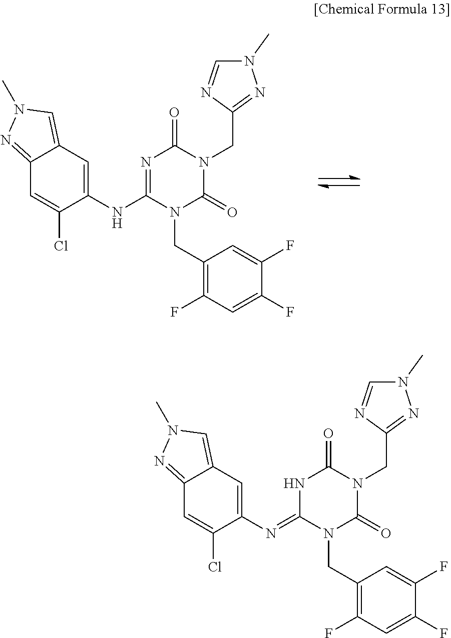 [SMILES: Cn1cnc(Cn2c(=O)nc(Nc3cc4cn(C)nc4cc3Cl)n(Cc3cc(F)c(F)cc3F)c2=O)n1 ; Cn1cnc(Cn2c(=O)[nH]/c(=N\c3cc4cn(C)nc4cc3Cl)n(Cc3cc(F)c(F)cc3F)c2=O)n1]

One or more hydrogen atom, carbon atom and/or another atom of the compound represented by Formula (I) may be replaced with an isotope of the hydrogen atom, carbon atom and/or another atom. Examples of such an isotope include hydrogen, carbon, nitrogen, oxygen, phosphorus, sulfur, fluorine, iodine, and chlorine such as ²H, ³H, ¹¹C, ¹³C, ⁴C, ¹⁵N, ¹⁸O, ¹⁷O, ³¹P, ³²P, ³⁵S, ¹⁸F, ¹²³I, and ³⁶Cl. The compound represented by Formula (I) also includes compounds replaced with such an isotope. The compounds replaced with an isotope are also useful as a pharmaceutical product and include all of radiolabeled forms of the compound represented by Formula (I).

Furthermore, the crystal of the compound represented by Formula (I) may be a deuterated form. The crystal of the compound represented by Formula (I) may be labeled with an isotopic element (for example, ³H, ¹⁴C, ³⁵S, ¹²⁵I, etc.).

The radiolabeled form of the compound represented by Formula (I) can be prepared by the method well known in this technical field. For example, a tritium-labeled compound represented by Formula (I) can be prepared by introducing tritium into a specific compound represented by Formula (I) by catalytic dehalogenation reaction using tritium. This method includes reaction of a precursor which is a compound represented by Formula (I) appropriately halogenated with tritium gas in the presence of an appropriate catalyst, for example, Pd/C, and in the presence or absence of a base. For another appropriate method for preparing a tritium-labeled compound, “Isotopes in the Physical and Biomedical Sciences, Vol. 1, Labeled Compounds (Part A), Chapter 6 (1987)” can be referred to. ¹⁴C-labeled compound can be prepared using a raw material having ¹⁴C carbon.

Examples of the pharmaceutically acceptable salt of the compound represented by Formula (I) include salts of the compound represented by Formula (I) with alkali metal (for example, lithium, sodium, potassium, etc.), alkaline earth metal (for example, calcium, barium, etc.), magnesium, transition metal (for example, zinc, iron, etc.), ammonia, organic base (for example, trimethylamine, triethylamine, dicyclohexylamine, ethanolamine, diethanolamine, triethanolamine, meglumine, ethylenediamine, pyridine, picoline, quinoline, etc.) and amino acid or salts of the compound represented by Formula (I) with inorganic acid (for example, hydrochloric acid, sulfuric acid, nitric acid, carbonic acid, hydrobromic acid, phosphoric acid, hydroiodic acid, etc.), and organic acid (for example, formic acid, acetic acid, propionic acid, trifluoroacetic acid, citric acid, lactic acid, tartaric acid, oxalic acid, maleic acid, fumaric acid, succinic acid, mandelic acid, glutaric acid, malic acid, benzoic acid, phthalic acid, ascorbic acid, benzenesulfonic acid, p-toluenesulfonic acid, methanesulfonic acid, ethanesulfonic acid, trifluoroacetic acid, etc.). These salts can be formed by the method which is usually performed.

In the medicament of the present invention, a complex of the compound represented by Formula (I) or a pharmaceutically acceptable salt thereof can be used. The compound represented by Formula (I) or a pharmaceutically acceptable salt thereof may form a solvate (for example, hydrate, etc.), a cocrystal and/or a clathrate, and these are described as “complex” herein.

In the “solvate” used herein, any number of solvent molecules (for example, water molecule, etc.) may be coordinated, for example, to the compound represented by Formula (I). By leaving the compound represented by Formula (I) or a pharmaceutically acceptable salt thereof in the atmosphere, it may absorb moisture to adhere with absorbed water or form a hydrate thereof.

Examples of the solvent molecule include acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethylene glycol, formamide, hexane, methanol, 2-methoxyethanol, methylbutyl ketone, methylcyclohexane, N-methylpyrrolidone, nitromethane, pyridine, sulfolane, tetralin, toluene, 1,1,2-trichloroethene, xylene, acetic acid, anisole, 1-butanol, 2-butanol, n-butyl acetate, t-butyl methyl ether, cumene, dimethylsulfoxide, ethyl acetate, diethyl ether, ethyl formate, formic acid, heptane, isobutyl acetate, isopropyl acetate, methyl acetate, 3-methyl-1-butanol, methyl ethyl ketone, methyl isobutyl ketone, 2-methyl-1-propanol, pentane, 1-pentanol, 1-propanol, 2-propanol, propyl acetate, tetrahydrofuran, water (that is, hydrate), ethanol, acetone, 1,1-diethoxypropane, 1,1-dimethoxymethane, 2,2-dimethoxypropane, iso-octane, isopropyl ether, methyl isopropyl ketone, methyltetrahydrofuran, petroleum ether, trichloroacetic acid, and trifluoroacetic acid, preferably, acetic acid, anisole, 1-butanol, 2-butanol, n-butyl acetate, t-butyl methyl ether, cumene, dimethylsulfoxide, ethyl acetate, diethyl ether, ethyl formate, formic acid, heptane, isobutyl acetate, isopropyl acetate, methyl acetate, 3-methyl-1-butanol, methyl ethyl ketone, methyl isobutyl ketone, 2-methyl-1-propanol, pentane, 1-pentanol, 1-propanol, 2-propanol, propyl acetate, tetrahydrofuran, water (that is, hydrate), ethanol, acetone, 1,1-diethoxypropane, 1,1-dimethoxymethane, 2,2-dimethoxypropane, iso-octane, isopropyl ether, methyl isopropyl ketone, methyltetrahydrofuran, petroleum ether, trichloroacetic acid, and trifluoroacetic acid, and more preferably, water (that is, hydrate), ethanol, acetone, 1,1-diethoxypropane, 1,1-dimethoxymethane, 2,2-dimethoxypropane, iso-octane, isopropyl ether, methyl isopropyl ketone, methyltetrahydrofuran, petroleum ether, trichloroacetic acid, and trifluoroacetic acid.

The “cocrystal” used herein means that counter molecules are regularly arranged in the same crystal lattice and may include any number of counter molecules. Further, the cocrystal indicates one in which the intermolecular interaction between the compound and the counter molecule is mediated with non-covalent and non-ionic chemical interaction such as hydrogen bonding or van der Waals' force.

For example, the cocrystal of the compound represented by Formula (I-B) may be composed of the compound represented by Formula (I-B) and a counter molecule and may include any number of counter molecules. Preferably, the cocrystal may be composed of the compound represented by Formula (I-B) and fumaric acid and may include any number of fumaric acids. Further preferably, the cocrystal is a cocrystal composed of the compound represented by Formula (I-B) and fumaric acid at a molar ratio of 1:1.

The cocrystal is distinguished from a salt in that the compound is essentially uncharged or neutral.

The cocrystal is distinguished from a hydrate or a solvate in that the counter molecule is not water or a solvent.

The “crystal” used herein means a solid in which constituent atoms, ions, molecules, etc. are three-dimensionally arranged with regularity, and is distinguished from a non-crystalline solid not having such a regular inner structure.

The crystal of the compound of the present invention may be a single crystal, a twin crystal, a polycrystal, and the like.

Further, in the “crystal”, there may be a “crystalline polymorphism” which has the same composition but has different arrangement in the crystal, and crystals including these are referred to as the “crystalline form”.

The crystalline form and the degree of crystallinity can be measured by many techniques including, for example, X-ray powder diffraction measurement, Raman spectroscopy, an infrared absorption spectrum measurement method, moisture adsorption-desorption measurement, differential scanning calorimetry, and dissolution properties.

Furthermore, a “crystalline polymorphism” may be formed by recrystallization of the compound represented by Formula (I), a pharmaceutically acceptable salt thereof, or the complex thereof.

In the medicament of the present invention, such various salts, complexes (hydrate, solvate, cocrystal, and clathrate), and the crystalline polymorphism can be used, and a mixture of two or more kinds thereof can also be used.

### (X-Ray Powder Diffraction (XRPD))

The X-ray powder diffraction (XRPD) is one of the most sensitive analytical methods for measuring the crystalline form and crystallinity of solid. When crystals are irradiated with X-rays, the X-rays are reflected by the crystal lattice planes and mutually interfere, and the ordered diffraction lines corresponding to the periodicity of the structure are observed. On the other hand, in the case of amorphous solids, usually, since they do not have the ordered iteration periodicity in the structure, diffraction phenomenon does not occur, and featureless broad XRPD patterns (also called halo patterns) are shown.

The crystalline form of the compound represented by Formula (I-B) can be identified by the X-ray powder diffraction pattern and characteristic diffraction peaks. The crystalline form of the compounds represented by Formula (I-B) can be distinguished from the other crystalline form by the presence of characteristic diffraction peaks.

The characteristic diffraction peaks used herein are peaks selected from the observed diffraction pattern. The characteristic diffraction peaks are selected from preferably about ten, more preferably about five, and further preferably about three in the diffraction pattern.

In order to distinguish between multiple crystals, a peak which is shown for the crystal and not shown for the other crystal becomes a more preferable characteristic peak than the intensity of a peak when the crystal is specified. The crystal can be characterized by one or two peak(s) if it is such characteristic peak(s). By comparing the chart obtained by measuring, if these characteristic peaks coincide, the X-ray powder diffraction pattern can be said to substantially match up.

Since an error in the range of ±0.2° may occur in diffraction angles (2θ) in X-ray powder diffraction, in general, the value of the diffraction angle of X-ray powder diffraction should be understood as the one including values in a range of around ±0.2°. Therefore, the compound of the present invention includes not only crystalline forms whose diffraction angles of the peaks in X ray powder diffraction perfectly match, but also crystalline forms whose diffraction angles of the peaks match within an error of around ±0.2°.

In general, it is known that the intensities of the peaks shown in the following tables and drawings may vary depending on a number of factors, for example, selected orientation effects of crystals in the X-ray beam, effect of coarse particle, purity of the material to be analyzed, or degree of crystallinity of the sample. Furthermore, the peak positions may also shift for variations in sample height. Further, measurements using a different wavelength will result in different shifts according to the Bragg equation (nλ=2d sin θ). Such another XRPD patterns obtained by using a different wavelength are also within the scope of the compound of the present invention.

### (Single Crystal Structural Analysis)

By one of methods of identifying a crystal, crystallographic parameters in the crystal, atomic coordinates (values indicating spatial positional relationship of individual atoms), and the three-dimensional structural model can be obtained. Refer to “Manual of X-ray structural analysis” written by Sakurai Toshio, published by Shokabo Co., Ltd. (1983), X-Ray Structure Determination: A Practical Guide, written by Stout & Jensen, Macmillan Co., New York (1968), and the like. The single crystal structural analysis is useful to identify the crystalline structures of the complex, salt, optical isomer, tautomer, and geometric isomer of the present invention.

### (Production Method of Compound Represented by Formula (I))

The compound represented by Formula (I) can be produced, for example, by a general synthesis method described below. Extraction, purification, and the like may be carried out by conventional methods practiced in organic chemistry experiments.

The compound represented by Formula (I) can be synthesized with reference to methods known in the art. The compound can be produced, for example, with reference to WO 2010092966 A, WO 2012020749 A, WO 2013089212 A, WO 2014200078 A, WO 2012020742 A, and WO 2013118855 A.

### (Method A)

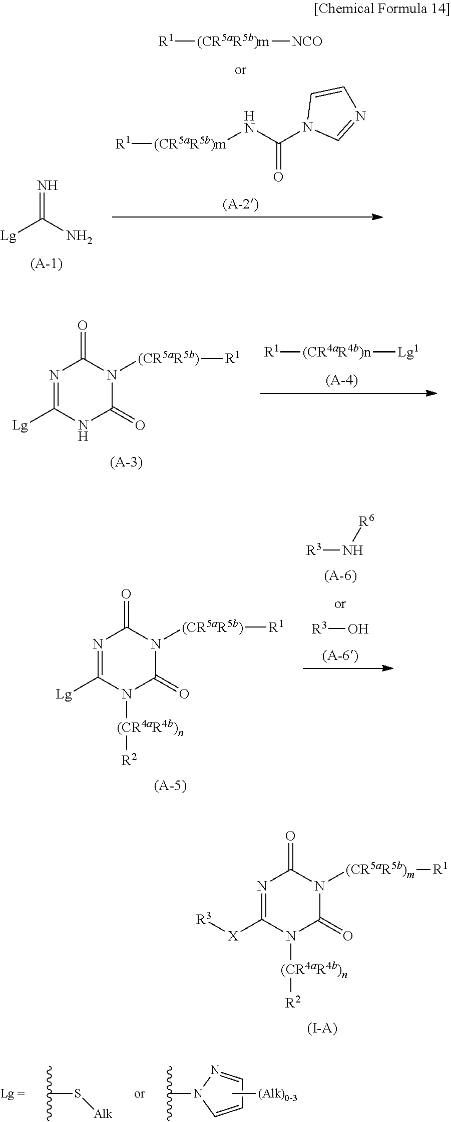

wherein Alk is C1-C3 alkyl, Lg¹ is a leaving group, R⁶ is a hydrogen atom, and other symbols are as defined above.

### (First Step)

Compound (A-1) or its hydrochloride or bromate, etc. is reacted with isocyanate (A-2) or 1-carbamoylimidazole (A-2′) in a solvent such as N,N-dimethylformamide, N,N-dimethylacetamide, N,N′-dimethylimidazolidinone, dimethylsulfoxide, or THF in the presence of a base such as DBU, triethylamine, N,N-diisopropylethylamine, or pyridine (preferably, DBU) at −20° C. to 50° C., preferably −10° C. to under ice-cooling. Subsequently, Compound (A-3) can be produced by reacting the reaction mixture with a carbonylating agent such as 1,1′-carbonyldiimidazole, phosgene, or triphosgene and a base such as DBU, triethylamine, N,N-diisopropylethylamine, or pyridine (preferably, DBU) at −20° C. to 50° C., preferably −10° C. to under ice-cooling.

### (Second Step)

Compound (A-5) can be produced by reacting Compound (A-3) with Compound (A-4) in a solvent such as acetonitrile, acetone, DMF, or DMSO in the presence of a base such as potassium carbonate, sodium carbonate, N,N-diisopropylethylamine, at 50° C. to under refluxing with heating, preferably under refluxing with heating.

Examples of the leaving group include halogen and —OSO₂(CₜF₂ₜ₊₁) (wherein t is an integer of 1 to 4). The halogen is preferably chlorine, iodine, and bromine, and the OSO₂(CₜF₂ₜ₊₁) group is preferably a —OTf group (trifluoromethanesulfonic acid ester).

### (Third Step)

A compound represented by Compound (I-A) can be produced by reacting Compound (A-5) with Compound (A-6) or Compound (A-6′) in a solvent such as NMP, DMF, DMA, DMSO, tert-butanol, or 2-methyl-2-butanol, in the presence or absence of an acid such as acetic acid at 60° C. to 150° C., preferably 80° C. to 120° C.

By using the optically active isocyanate (A-2), a compound represented by Compound (I-A) which is optically active can be produced.

(Method D) Case where Y is N and m is 0

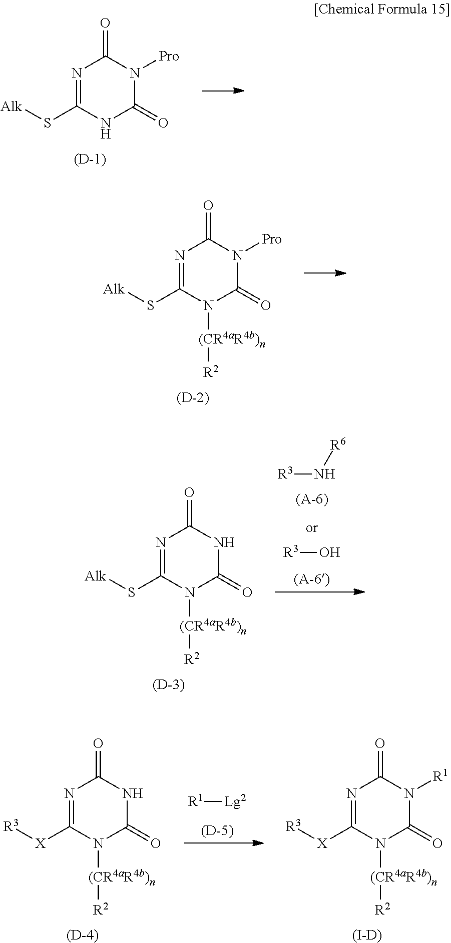

wherein Alk is C1-C3 alkyl, Pro is C1-C4 alkyl or tert-butoxycarbonyl, Lg² is a leaving group, R⁶ is a hydrogen atom, and other symbols are as defined above.

### (First Step)

Compound (D2) can be produced from Compound (D-1) in the same manner as in the second step of Method A described above.

### (Second Step)

Compound (D-3) can be produced by treating Compound (D-2) at −20° C. to room temperature, preferably at room temperature, with a strong acid such as TFA in the presence or absence of an organic solvent.

### (Third Step)

Compound (D-4) can be produced from Compound (D-3) in the same manner as in the third step of Method A described above.

### (Fourth Step)

Compound (I-D) can be produced by Goldberg amination reaction using Compound (D-4) and Compound (D-5).

As a leaving group, the leaving group described in Step 1 of Method A is exemplified.

As a catalyst, for example, commercially available copper catalysts such as copper iodide, copper cyanide, and copper bromide can be used.

As a ligand, 1,2-dimethylethylenediamine, trans-N,N′-dimethylcyclohexane-1,2-diamine, and the like can be used.

As a base, potassium carbonate, potassium phosphate, and the like can be used.

As a solvent, NMP, dioxane, DMSO, and the like can be used.

Regarding the reaction temperature, the reaction may be performed in the range of room temperature to the reflux temperature of the solvent, and preferably may be performed under refluxing with heating.

(Method E) Case where m is 1

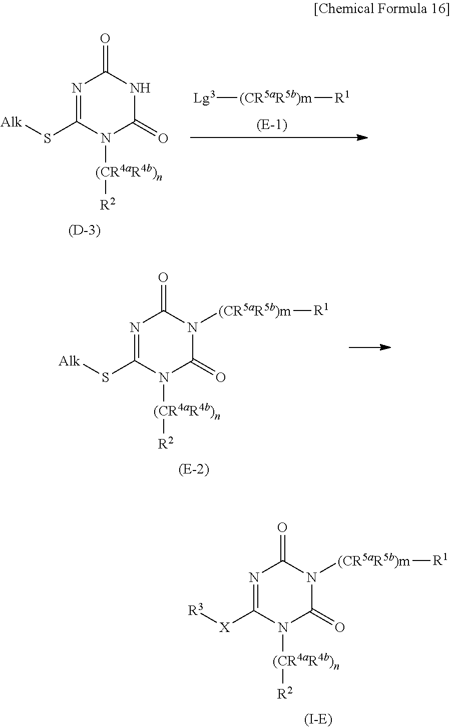

wherein Alk is C1-C3 alkyl, Lg³ is a leaving group, and other symbols are as defined above.

### (First Step)

Compound (E-2) can be produced in the same manner as in the second step of Method A described above.

As a leaving group, the leaving group described in Step 1 of Method A is exemplified.

### (Second Step)

The compound represented by Compound (I-E) can be produced in the same manner as in the third step of Method A described above.

The compound represented by (A) in the medicament of the present invention has coronavirus 3CL protease inhibitory activity, and thus is useful as a therapeutic and/or prophylactic agent for virus diseases.

Further, the compound represented by (A) in the medicament of the present invention has usefulness as a medicament and has preferably any or a plurality of the following superior properties.
a) The compound has weak inhibitory action against CYP enzymes (for example, CYP1A2, CYP2C9, CYP2C19, CYP2D6, CYP3A4, etc.).
b) The compound shows excellent pharmacokinetics such as high bioavailability or moderate clearance.
c) The compound has high metabolic stability.
d) The compound does not show irreversible inhibitory action against CYP enzymes (for example, CYP3A4) in the range of the concentration of the measurement conditions described herein.
e) The compound does not show mutagenesis.
f) The compound has a low risk of cardiovascular systems.
g) The compound shows high solubility.
h) The compound shows a high protein unbinding ratio (fu value).
i) The compound has high coronavirus 3CL protease selectivity.
j) The compound has high coronavirus proliferation inhibitory activity. For example, the compound has high coronavirus proliferation inhibitory activity with addition of human serum (HS) or human serum albumin (HSA).

Furthermore, the salt-crystal-complex (cocrystal) of the compound represented by (A) in the medicament of the present invention has usefulness as a medicament and has preferably any or a plurality of the following superior properties.
bb) It shows excellent pharmacokinetics such as high bioavailability, moderate clearance, high AUC, or high maximum drug concentration.
gg) It shows high solubility, high chemical stability, and low moisture absorbency.

Commercially available products can also be used as (B) in the medicament of the present invention, and (B) can be prepared, for example, by methods known in the art such as methods described in Non-patent Document 14 (describing the preparation method of PF-07321332, etc.), WO 2005113580 A (describing the preparation of a 3CL protease inhibitor), etc.

As an embodiment, the present invention provides a medicament characterized by combining
(A) a compound represented by Formula (I) or a pharmaceutically acceptable salt thereof; and
(B) a COVID-19 exacerbation suppressant (provided that, excluding the compound represented by Formula (I) or a pharmaceutically acceptable salt thereof).

As another embodiment, the present invention provides a medicament for suppression of exacerbation of COVID-19, comprising (A) and (B) in combination.

The term “medicament characterized by combination” herein includes a medicament comprising each compound, an embodiment in which each compound is used as a combination drug, an embodiment in which each compound is used as a kit, an embodiment in which it is administered simultaneously, an embodiment in which it is administered at intervals, and an embodiment in which a certain medicament is used in combination with another medicament. Although there are also cases where the expression “combining (combined)” will be omitted, these cases have the same meaning.

The compound represented by Formula (I) or a pharmaceutically acceptable salt thereof of (A) can be used in combination with (B) and its action can be enhanced.

As an embodiment, the present invention provides a COVID-19 exacerbation suppression enhancer for (B), comprising (A).

As an embodiment, the present invention provides a COVID-19 exacerbation suppression enhancer for (A), comprising (B).

As a preferable embodiment, there is a medicament characterized by combining the following (A) and (B).
(A) is the compound represented by the above (a-1), (a-2), (a-3), or (a-4) or a pharmaceutically acceptable salt thereof, or a compound represented by formula:

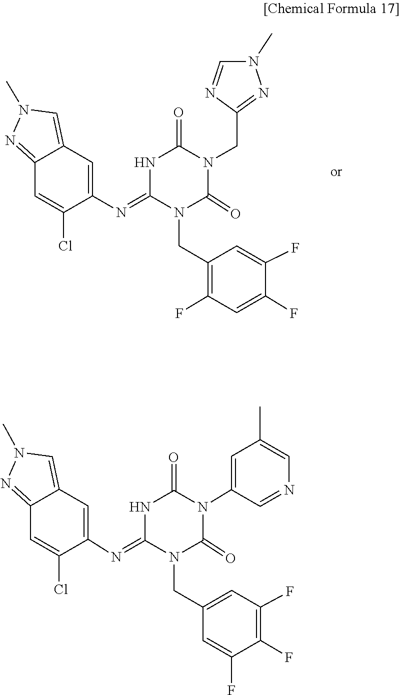 [SMILES: Cn1cnc(Cn2c(=O)[nH]/c(=N\c3cc4cn(C)nc4cc3Cl)n(Cc3cc(F)c(F)cc3F)c2=O)n1 ; Cc1cncc(-n2c(=O)[nH]/c(=N\c3cc4cn(C)nc4cc3Cl)n(Cc3cc(F)c(F)c(F)c3)c2=O)c1]

or a pharmaceutically acceptable salt thereof.
(B) is at least one selected from the group consisting of an anti-SARS-CoV-2 monoclonal antibody, an RNA-dependent RNA polymerase inhibitor, a 3CL protease inhibitor (provided that, excluding the compound of (A) or a pharmaceutically acceptable salt thereof), and a TMPRSS2 inhibitor.

As a preferable embodiment, there is a medicament characterized by combining the following (A) and (B).
(A) is the compound represented by the above (a-1), (a-2), (a-3), or (a-4) or a pharmaceutically acceptable salt thereof, or a compound represented by formula:

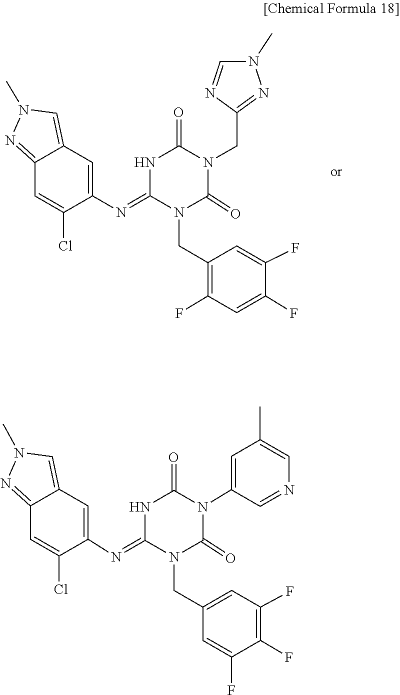 [SMILES: Cn1cnc(Cn2c(=O)[nH]/c(=N\c3cc4cn(C)nc4cc3Cl)n(Cc3cc(F)c(F)cc3F)c2=O)n1 ; Cc1cncc(-n2c(=O)[nH]/c(=N\c3cc4cn(C)nc4cc3Cl)n(Cc3cc(F)c(F)c(F)c3)c2=O)c1]

or a pharmaceutically acceptable salt thereof.
(B) is at least one antibody or compound or a pharmaceutically acceptable salt thereof selected from (i) or (ii):
(i) Casirivimab, Imdevimab, Sotrovimab, Tixagevimab, Cilgavimab, or Bebtelovimab,
(ii) Molnupiravir, Remdesivir, AT-527, PF-07321332, PF-00835231, GC376, or Camostat, or a pharmaceutically acceptable salt thereof.

As another embodiment, there is a medicament characterized by combining the following (A) and (B).
(A) is the compound represented by the above (a-4) or a pharmaceutically acceptable salt thereof.
(B) is at least one selected from the group consisting of an anti-SARS-CoV-2 monoclonal antibody, an RNA-dependent RNA polymerase inhibitor, a 3CL protease inhibitor (provided that, excluding the compound of (A) or a pharmaceutically acceptable salt thereof), and a TMPRSS2 inhibitor.

As another embodiment, there is a medicament characterized by combining the following (A) and (B).
(A) is the compound represented by the above (a-4) or a pharmaceutically acceptable salt thereof.
(B) is an anti-SARS-CoV-2 monoclonal antibody.

As another embodiment, there is a medicament characterized by combining the following (A) and (B).
(A) is the compound represented by the above (a-4) or a pharmaceutically acceptable salt thereof.
(B) is at least one antibody or compound or a pharmaceutically acceptable salt thereof selected from (i) or (ii):
(i) Casirivimab, Imdevimab, Sotrovimab, Tixagevimab, Cilgavimab, or Bebtelovimab,
(ii) Molnupiravir, Remdesivir, AT-527, PF-07321332, PF-00835231, GC376, or Camostat, or a pharmaceutically acceptable salt thereof.

As another embodiment, there is a medicament characterized by combining the following (A) and (B).
(A) is the compound represented by the above (a-3) or a pharmaceutically acceptable salt thereof.
(B) is at least one selected from the group consisting of an anti-SARS-CoV-2 monoclonal antibody, an RNA-dependent RNA polymerase inhibitor, a 3CL protease inhibitor (provided that, excluding the compound of (A) or a pharmaceutically acceptable salt thereof), and a TMPRSS2 inhibitor.

As another embodiment, there is a medicament characterized by combining the following (A) and (B).
(A) is the compound represented by the above (a-3) or a pharmaceutically acceptable salt thereof.
(B) is an anti-SARS-CoV-2 monoclonal antibody.

As another embodiment, there is a medicament characterized by combining the following (A) and (B).
(A) is the compound represented by the above (a-3) or a pharmaceutically acceptable salt thereof.
(B) is at least one antibody or compound or a pharmaceutically acceptable salt thereof selected from (i) or (ii):
(i) Casirivimab, Imdevimab, Sotrovimab, Tixagevimab, Cilgavimab, or Bebtelovimab,
(ii) Molnupiravir, Remdesivir, PF-07321332, GC376, or Camostat, or a pharmaceutically acceptable salt thereof.

As another embodiment, there is a medicament characterized by combining the following (A) and (B).
(A) is the compound represented by the above (a-2) or a pharmaceutically acceptable salt thereof.
(B) is at least one selected from the group consisting of an anti-SARS-CoV-2 monoclonal antibody, an RNA-dependent RNA polymerase inhibitor, a 3CL protease inhibitor (provided that, excluding the compound of (A) or a pharmaceutically acceptable salt thereof), and a TMPRSS2 inhibitor.

As another embodiment, there is a medicament characterized by combining the following (A) and (B).
(A) is the compound represented by the above (a-2) or a pharmaceutically acceptable salt thereof.
(B) is an anti-SARS-CoV-2 monoclonal antibody.

As another embodiment, there is a medicament characterized by combining the following (A) and (B).
(A) is the compound represented by the above (a-2) or a pharmaceutically acceptable salt thereof.
(B) is at least one antibody or compound or a pharmaceutically acceptable salt thereof selected from (i) or (ii):
(i) Casirivimab, Imdevimab, Sotrovimab, Tixagevimab, Cilgavimab, or Bebtelovimab,
(ii) Molnupiravir, Remdesivir, PF-07321332, GC376, or Camostat, or a pharmaceutically acceptable salt thereof.

As another embodiment, there is a medicament characterized by combining the following (A) and (B).
(A) is the compound represented by the above (a-1) or a pharmaceutically acceptable salt thereof.
(B) is at least one selected from the group consisting of an anti-SARS-CoV-2 monoclonal antibody, an RNA-dependent RNA polymerase inhibitor, a 3CL protease inhibitor (provided that, excluding the compound of (A) or a pharmaceutically acceptable salt thereof), and a TMPRSS2 inhibitor.

As another embodiment, there is a medicament characterized by combining the following (A) and (B).
(A) is the compound represented by the above (a-1) or a pharmaceutically acceptable salt thereof.
(B) is an anti-SARS-CoV-2 monoclonal antibody.

As another embodiment, there is a medicament characterized by combining the following (A) and (B).
(A) is the compound represented by the above (a-1) or a pharmaceutically acceptable salt thereof.
(B) is at least one antibody or compound or a pharmaceutically acceptable salt thereof selected from (i) or (ii):
(i) Casirivimab, Imdevimab, Sotrovimab, Tixagevimab, Cilgavimab, or Bebtelovimab,
(ii) Molnupiravir, Remdesivir, PF-07321332, GC376, or Camostat, or a pharmaceutically acceptable salt thereof.

As another embodiment, there is a medicament characterized by combining the following (A) and (B).
(A) is a compound represented by formula:

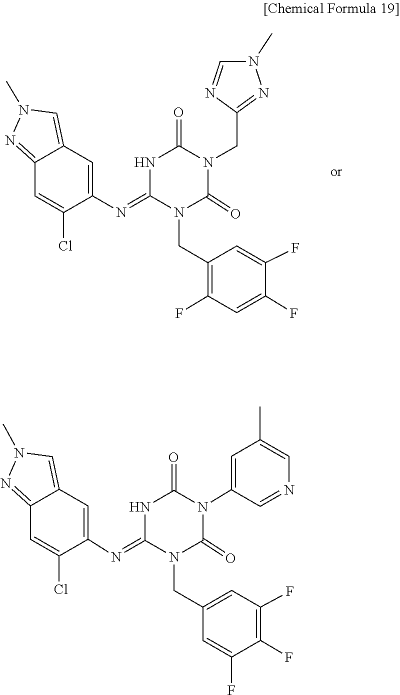 [SMILES: Cn1cnc(Cn2c(=O)[nH]/c(=N\c3cc4cn(C)nc4cc3Cl)n(Cc3cc(F)c(F)cc3F)c2=O)n1 ; Cc1cncc(-n2c(=O)[nH]/c(=N\c3cc4cn(C)nc4cc3Cl)n(Cc3cc(F)c(F)c(F)c3)c2=O)c1]

or a pharmaceutically acceptable salt thereof.
(B) is at least one selected from the group consisting of an anti-SARS-CoV-2 monoclonal antibody, an RNA-dependent RNA polymerase inhibitor, a 3CL protease inhibitor (provided that, excluding the compound of (A) or a pharmaceutically acceptable salt thereof), and a TMPRSS2 inhibitor.

As another embodiment, there is a medicament characterized by combining the following (A) and (B).
(A) is a compound represented by formula:

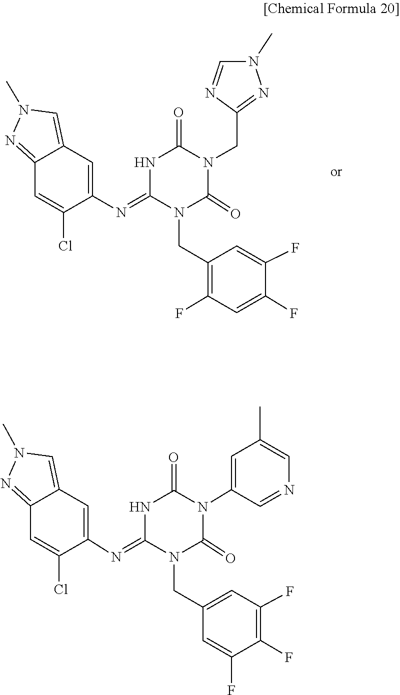 [SMILES: Cn1cnc(Cn2c(=O)[nH]/c(=N\c3cc4cn(C)nc4cc3Cl)n(Cc3cc(F)c(F)cc3F)c2=O)n1 ; Cc1cncc(-n2c(=O)[nH]/c(=N\c3cc4cn(C)nc4cc3Cl)n(Cc3cc(F)c(F)c(F)c3)c2=O)c1]

or a pharmaceutically acceptable salt thereof.
(B) is an anti-SARS-CoV-2 monoclonal antibody.

As another embodiment, there is a medicament characterized by combining the following (A) and (B).
(A) is a compound represented by formula:

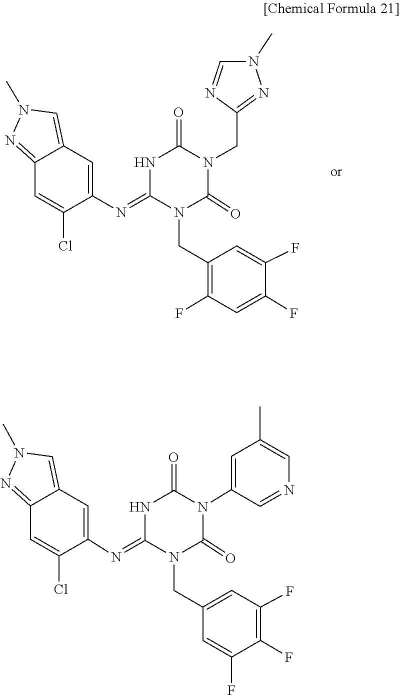 [SMILES: Cn1cnc(Cn2c(=O)[nH]/c(=N\c3cc4cn(C)nc4cc3Cl)n(Cc3cc(F)c(F)cc3F)c2=O)n1 ; Cc1cncc(-n2c(=O)[nH]/c(=N\c3cc4cn(C)nc4cc3Cl)n(Cc3cc(F)c(F)c(F)c3)c2=O)c1]

or a pharmaceutically acceptable salt thereof.
(B) is at least one antibody or compound or a pharmaceutically acceptable salt thereof selected from (i) or (ii):
(i) Casirivimab, Imdevimab, Sotrovimab, Tixagevimab, Cilgavimab, or Bebtelovimab,
(ii) Molnupiravir, Remdesivir, PF-07321332, GC376, or Camostat, or a pharmaceutically acceptable salt thereof.

As another embodiment, there is a medicament characterized by combining the following (A) and (B).
(A) is a compound represented by Formula (I-B):

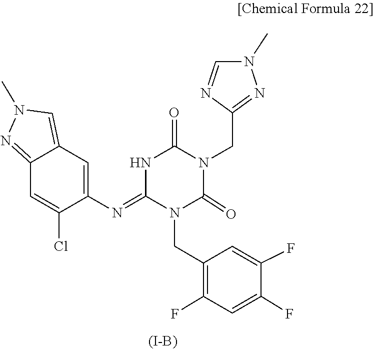 [SMILES: Cn1cnc(Cn2c(=O)[nH]/c(=N\c3cc4cn(C)nc4cc3Cl)n(Cc3cc(F)c(F)cc3F)c2=O)n1 ; "(I-B)"]

or a pharmaceutically acceptable salt thereof.
(B) is at least one selected from the group consisting of an anti-SARS-CoV-2 monoclonal antibody, an RNA-dependent RNA polymerase inhibitor, a 3CL protease inhibitor (provided that, excluding the compound of (A) or a pharmaceutically acceptable salt thereof), and a TMPRSS2 inhibitor.

As another embodiment, there is a medicament characterized by combining the following (A) and (B).
(A) is a compound represented by Formula (I-B):

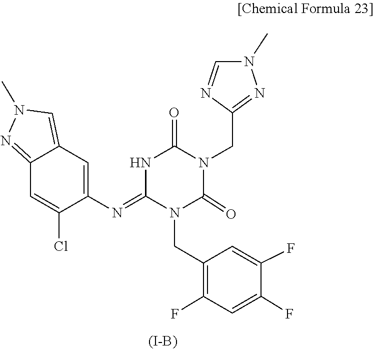 [SMILES: Cn1cnc(Cn2c(=O)[nH]/c(=N\c3cc4cn(C)nc4cc3Cl)n(Cc3cc(F)c(F)cc3F)c2=O)n1 ; "(I-B)"]

or a pharmaceutically acceptable salt thereof.
(B) is an anti-SARS-CoV-2 monoclonal antibody.

As another embodiment, there is a medicament characterized by combining the following (A) and (B).
(A) is a compound represented by Formula (I-B):

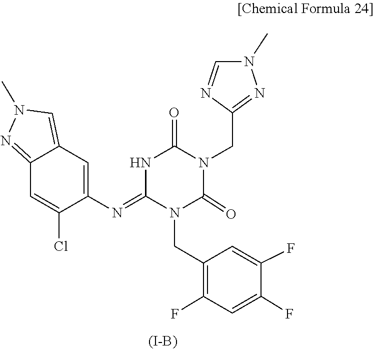 [SMILES: Cn1cnc(Cn2c(=O)[nH]/c(=N\c3cc4cn(C)nc4cc3Cl)n(Cc3cc(F)c(F)cc3F)c2=O)n1 ; "(I-B)"]

or a pharmaceutically acceptable salt thereof.
(B) is at least one antibody or compound or a pharmaceutically acceptable salt thereof selected from (i) or (ii):
(i) Casirivimab, Imdevimab, Sotrovimab, Tixagevimab, Cilgavimab, or Bebtelovimab,
(ii) Molnupiravir, Remdesivir, PF-07321332, GC376, or Camostat, or a pharmaceutically acceptable salt thereof.

The medicament of the present invention can also be administered orally or parenterally. Examples of methods for parenteral administration include dermal, subcutaneous, intravenous, intraarterial, intramuscular, intraperitoneal, transmucosal, inhalation, transnasal, ophthalmic, inner ear or vaginal administration, and the like.

In the case of oral administration, any forms, which are usually used, such as oral solid formulations (for example, tablets, powders, granules, capsules, pills, films, etc.), oral liquid formulations (for example, suspension, emulsion, elixir, syrup, limonade, spirit, aromatic water, extract, decoction, tincture, etc.) and the like may be prepared according to the usual method and administered. The tablets may be sugar-coated tablets, film-coated tablets, enteric-coating tablets, sustained-release tablets, troche tablets, sublingual tablets, buccal tablets, chewable tablets, or orally disintegrated tablets, powders and granules may be dry syrups, and capsules may be soft capsules, micro capsules, or sustained-release capsules.

In the case of parenteral administration, any forms, which are usually used, such as injections, infusion, external preparations (for example, ear drops, nasal drops, eye drops, aerosol, inhalation, lotion, injection agents, coating agents, mouthwash, enemas, ointments, plasters, jellies, creams, patches, cataplasms, external powders, suppositories, etc.) and the like can be preferably administrated. The injections may be emulsions whose type is O/W, W/O, O/W/O, W/O/W, or the like.

The pharmaceutical composition can be manufactured by mixing an effective amount of the medicament of the present invention with various pharmaceutical additives suitable for the formulation, such as excipients, binders, disintegrants, and lubricants, as necessary. Further, the pharmaceutical composition can also be used for pediatric patients, geriatric patients, serious cases, or operations by appropriately changing the effective amount of the compound in the medicament of the present invention, formulation and/or various pharmaceutical additives. For example, pediatric pharmaceutical compositions may be administered to patients who are neonates (younger than 4 weeks old after the birth), infants (4 weeks old to younger than 1 year old after the birth), infant children (1 year old or older and younger than 7 years old), children (7 years old or older and younger than 15 years old), or 15 to 18 years old. For example, the geriatric pharmaceutical compositions may be administered to patients who are 65 years old or older.

Although it is desirable to set the dose of the pharmaceutical composition comprising the compound represented by (A) in the medicament of the present invention (for example, the pharmaceutical composition containing fumaric acid cocrystal Form I of the compound represented by Formula (I-B)) in consideration of the age and body weight of the patient, disease type and degree or administration route, and the like, the dose in the case of orally administration is within the range of usually 0.05 to 200 mg/kg/day and preferably 0.1 to 100 mg/kg/day. Although varying depending on the administration route, the dose in the case of parenteral administration is within the range of usually 0.005 to 200 mg/kg/day and preferably 0.01 to 100 mg/kg/day. It may be administered once to several times a day.

The dose of the medicament of the present invention can be appropriately selected on the basis of the dose used on clinical. Furthermore, the mixing ratio of the compound represented by (A) and the concomitant medicament (B) can be appropriately selected in consideration of the subject of administration, administration route, target diseases, symptoms, combinations, and the like. For example, when the subject of administration is human, the concomitant medicament (B) may be used in the range of 0.001 to 1000 parts by weight with respect to 1 part by weight of the compound represented by (A).

The medicament or the enhancer of the present invention is used for treating and/or preventing coronavirus disease, particularly, infective disease due to SARS-CoV-2.

In an embodiment, SARS-CoV-2 causing infective disease to be targeted by the medicament or the enhancer of the present invention is a low sensitive virus. The low sensitivity also includes viruses exhibiting sensitivity only at a higher concentration than a level of the concentration to be inherently expected, in addition to narrow-sense resistant viruses (that is, viruses in which an antiviral drug is failed). The low sensitivity is usually determined by comparison with a criterial strain in an arbitrary measurement system of the antiviral agent, and can be measured, for example, using plaque reduction assay.

In an embodiment, the medicament or the enhancer of the present invention is used for an immune-compromised patient. Herein, the immune-compromised patient is determined as follows. That is, the immune strength can be measured by collecting a small amount of blood and examining the type, ratio, function, etc. of lymphocytes of third to tenth items. Typically, the immune strength can be determined by the number of white blood cells, and a case where the number of white blood cells is lower than the numerical range of a normal person can be determined to a low immune strength. The following values are normal values, and a case where a value is lower than the following values can be positioned as an immune-compromised case.

### (Standard Value)

Adults 4,000 to 9,000/μL
Children 6,000 to 10,000/μL
Infant children 6,000 to 18,000/μL
Neonates 9,000 to 25,000/μL

In an embodiment, the medicament or the enhancer for (A) of the present invention is characterized by reducing an emergence frequency of a low sensitive virus of (A).

In an embodiment, the medicament or the enhancer of the present invention is used for an adult aged 50 years or older. In a preferred embodiment, the medicament or the enhancer of the present invention is used for an adult aged 65 years or older. In a preferred embodiment, the medicament or the enhancer of the present invention is used for an adult aged 75 years or older.

The medicament or the enhancer of the present invention is used for a non-vaccinated patient against SARS-CoV-2. The reason for this is that the non-vaccinated patient against SARS-CoV-2 is assumed as one of so-called high-risk groups.

In an embodiment, the present invention is further used for a patient that falls into one or more categories of
(i) 50 years old or older,
(ii) obesity (for example, BMI >30 kg/m² or more),
(iii) cardiovascular disease (for example, including high-blood pressure),
(iv) asthma or chronic pulmonary disease,
(v) Type 1 or 2 diabetes,
(vi) chronic renal impairment (for example, including dialyzed patients),
(vii) chronic hepatic disease,
(viii) immunosuppressed state (for example, malignancy treatment, bone marrow or organ transplantation, immune deficiency, uncontrolled HIV, AIDS, sickle-cell anemia, thalassemia, long-term administration of an immunosuppressant, etc.),
(ix) chronic obstructive pulmonary disease (COPD),
(x) hyperlipidemia,
(xi) smoking,
(xii) immune deficiency after solid organ transplantation,
(xiii) pregnancy,
(xiv) a patient having a neurodevelopmental disease or complicated clinical conditions (for example, cerebral palsy, congenital disease, etc.), and
(xv) a patient having a high degree of medical dependence (for example, tracheostomy, gastric fistula, positive pressure ventilation, etc.).

These groups may be referred to as a high-risk group, but are not limited thereto.

As another embodiment, the medicament or the enhancer of the present invention is used for a patient having pneumonia caused by SARS-CoV-2.

In another embodiment, the medicament or the enhancer of the present invention is further used for a patient that falls into at least one of
(i) a patient with installation of extracorporeal membrane oxygenation (ECMO),
(ii) a patient with installation of inhalator,
(iii) a patient in ICU, and
(iv) a patient having an oxygen saturation (SpO₂) of 93% (room air) or less or requiring oxygen inhalation.

In another embodiment, the medicament or the enhancer of the present invention is further used for a patient that falls into at least one of
(i) an oxygen saturation (SpO₂) of less than 94% (room air, sea level),
(ii) PaO₂/FiO₂ of less than 300 mmHg,
(iii) a respiration rate of 30 or more/min, and
(iv) pulmonary infiltration of 50% or more.

The medicament, the enhancer, the kit, etc. of the present invention can include attachment and label which are described for providing instructions to paramedical personnel undertaking prevention or treatment such as physicians, for specification of a patient or a subject targeted by the present invention as a therapeutic objective, and guidelines for treatment and/or prevention such as dosage and administration, and precautions. However, these public documents are not limited to paper media, but can be provided through the Internet, and guidelines for prevention or treatment can be provided to physicians and the like on the basis of various other information sources in addition to public documents. Therefore, it should be understood that the present invention also includes embodiments that are used on the basis of information other than attachment and label.

In still another aspect, the present invention provides a method for preventing and/or treating COVID-19 or a method for enhancing COVID-19 exacerbation suppression action, having one or more features described herein and including a step of administering an effective amount of (A) and (B) to a subject in need thereof.

In another aspect, the present invention provides use of a combination of (A) and (B) in production of a medicament for preventing and/or treating COVID-19 or for COVID-19 exacerbation suppression action, having one or more features described herein.

### EXAMPLES

The present invention will be described in more detail below by way of Examples and Reference Examples, as well as Test Examples; however, the present invention is not limited thereto.

Furthermore, the meaning of each abbreviation used herein is as follows.
Boc: tert-butoxycarbonyl
CDI: carbonyldiimidazole
DBU: 1,8-diazabicyclo[5.4.0]-7-undecene
DIEA: N,N-diisopropylethylamine
DMA: N,N-dimethylacetamide
DMF: N,N-dimethylformamide
DMSO: dimethyl sulfoxide
DTT: dithiothreitol
EDC: 1-ethyl-3-(3-dimethylaminopropyl)carbodiimide
EDT: 1,2-ethanedithiol
EDTA: ethylenediaminetetraacetic acid
FBS: fetal bovine serum
HOBT: 1-hydroxybenzotriazole
LHMDS: lithium bis(trimethylsilyl)amide
MEM: Eagle's minimum essential medium
NMP: N-methylpyrrolidone
Pd(OAc)₂: palladium acetate
TFA: trifluoroacetic acid
TMSCl: chlorotrimethylsilane
Xantphos: 4,5′-bis(diphenylphosphino)-9,9′-dimethylxanthene
mM: mmol/L
μM: μmol/L
nM: nmol/L

### (Identification of Compounds)

NMR analysis of each Example was performed by 400 MHz using DMSO-d₆, CDCl₃, and Methanol-d₄. Furthermore, in NMR data shown in Examples and Reference Examples, not all measured peaks may be described.

“RT” herein means retention time in LC/MS: liquid chromatography/mass spectrometry and measured under the following conditions.

### (Measurement Conditions 1)

Column: ACQUITY UPLC (registered trademark) BEH C18 (1.7 μm i.d.2.1×50 mm) (Waters)
Flow rate: 0.8 mL/min
UV detection wavelength: 254 nm
Mobile phases: [A] is a 0.1% formic acid-containing aqueous solution, and [B] is a 0.1% formic acid-containing acetonitrile solution
Gradient: Linear gradient of 5% to 100% solvent [B] for 3.5 minutes was performed, and then 100% solvent [B] was maintained for 0.5 minutes.

### (Measurement Conditions 2)

Column: Shim-pack XR-ODS (2.2 μm, i.d.3.0×50 mm) (Shimadzu)
Flow rate: 1.6 mL/min
UV detection wavelength: 254 nm
Mobile phases: [A] is a 0.1% formic acid-containing aqueous solution, and [13] is a 0.1% formic acid-containing acetonitrile solution
Gradient: Linear gradient of 10% to 100% solvent [B] for 3 minutes was performed, and 100% solvent [B] was maintained for 0.5 minutes.

Note that, herein, the description MS (m/z) indicates the value observed in the mass spectrometry.

### (X-Ray Powder Diffraction Pattern Measurement)

X-ray powder diffraction pattern measurement of crystals obtained in each Example was performed according to a powder X-ray diffraction measurement method described in General Tests in Japanese Pharmacopoeia. Measurement conditions are as follows.

### (Device)

SmartLab manufactured by Rigaku Corporation

### (Operation Method)

Measuring method: reflection method
Wavelength used: CuKα ray
Tube current: 200 mA
Tube voltage: 45 kV
Sampling plate: aluminum
X-ray incident angle: 2.5°
Sampling width: 0.02°
Detector: HyPix-3000 (two-dimensional detection mode)

### (Measurement of Single Crystal Structural Analysis and Analysis Method)

The measurement conditions of the single crystal structural analysis and the analysis method are as follows.

### (Device)

XtaLAB P200 MM007 manufactured by Rigaku Corporation

### (Measurement Conditions)

Measurement temperature: 25° C.
Wavelength used: CuKα ray (λ=1.5418 Å)
Software: CrysAlisPro 1.171.39.46e (Rigaku Oxford Diffraction, 2018)

### (Data Processing)

Software: CrysAlisPro 1.171.39.46e (Rigaku Oxford Diffraction, 2018)

The data were corrected for the Lorentz, polarization and absorption effects.

### (Crystal Structural Analysis)

The phase determination was performed by using the direct method program ShelXT (Sheldrick, G. M., 2015), and the structural refinement by full-matrix least-square method was then performed by using ShelXL (Sheldrick, G. M., 2015). All temperature factors of non-hydrogen atoms were refined with anisotropic parameters. Hydrogen atoms were placed by calculation using default parameters of ShelXL and regarded as riding atom. All hydrogen atoms were refined with isotropic parameters.

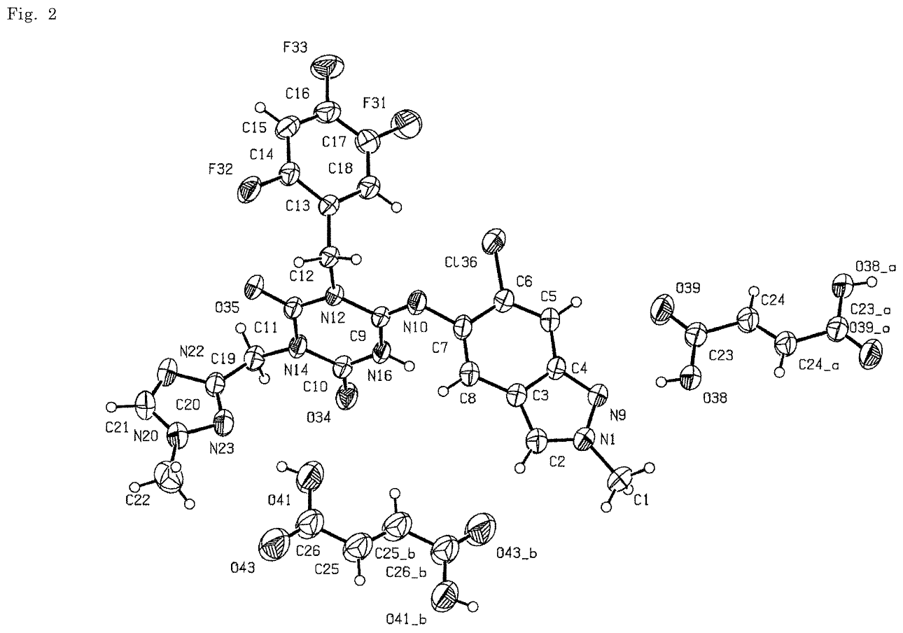

FIG. 2 was made using PLATON (Spek, 1991)/ORTEP (Johnson, 1976).

### Example 1

### Synthesis of Compound (I-003)

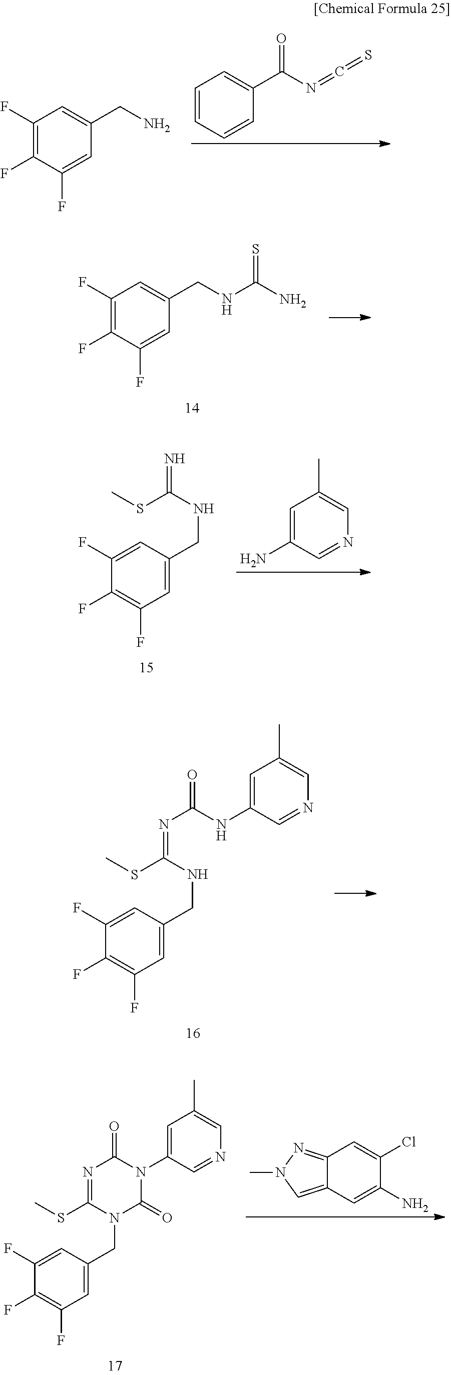

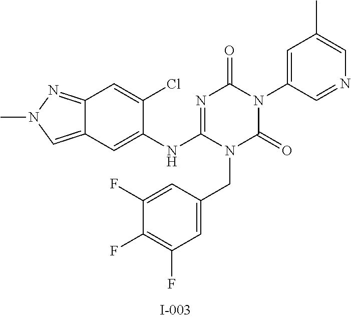 [SMILES: Cc1cncc(-n2c(=O)nc(Nc3cc4cn(C)nc4cc3Cl)n(Cc3cc(F)c(F)c(F)c3)c2=O)c1]

### Step 1 Synthesis of Compound 14

3,4,5-trifluorobenzylamine (3.34 g, 20.7 mmol) was dissolved in dichloromethane (33.4 mL) and cooled in a water bath. Benzoyl isothiocyanate (2.93 mL, 21.8 mmol) was added to the reaction solution and stirred at room temperature for 30 minutes.

The solvent was distilled, the residue was diluted with methanol, and a 1 mol/L aqueous solution of sodium oxide (7.45 mL, 7.45 mmol) was added. The reaction solution was stirred at room temperature for 30 minutes and a 2 mol/L aqueous solution of hydrochloric acid was added. The aqueous layer was extracted with ethyl acetate, and the organic layer was washed with a saturated sodium hydrogen carbonate aqueous solution and saturated saline. The organic layer was dried with sodium sulfate, and the solvent was distilled under reduced pressure, thereby obtaining a crude product (8.3 g) of Compound 14. This crude product was used for the next step without further purification, assuming that the yield was 100%.

LC/MS (ESI): m/z=221, RT=1.45 min, LC/MS measurement conditions 2

### Step 2 Synthesis of Compound 15

The crude product (8.3 g) of Compound 14, DMF (85 mL), and methyl iodide (4.84 mL, 77 mmol) were mixed, and the reaction solution was stirred at 50° C. for 40 minutes. Water was added to the reaction solution, extracted with ethyl acetate, and washed with water. A 2 mol/L aqueous solution of sodium oxide was added to the aqueous layer, and the aqueous layer was extracted with ethyl acetate. The organic layer was washed with water and saturated saline and dried with sodium sulfate. The solvent was distilled under reduced pressure, thereby obtaining a crude product (3.86 g, 16.5 mmol, yield 80%) of Compound 15.

LC/MS (ESI): m/z=235, RT=0.84 min, LC/MS measurement conditions 2

### Step 3 Synthesis of Compound 16

Triphosgene (0.507 g, 1.71 mmol) and THF (6 mL) were mixed, and the reaction solution was cooled in an ice bath. 3-Amino-5-methylpyridine (0.462 g, 4.27 mmol) and triethylamine (1.48 mL, 10.7 mmol) were mixed in THF (6 mL), and the solution thus obtained was added dropwise to the reaction solution. The reaction solution was stirred at room temperature for 40 minutes and then cooled in an ice bath. Compound 15 (1 g, 4.27 mmol) was added to the reaction solution and stirred at room temperature for 55 minutes. Water was added, the aqueous layer was extracted with ethyl acetate, and the organic layer was washed with water. The organic layer was dried with magnesium sulfate, and the solvent was distilled under reduced pressure, thereby obtaining a crude product (1.57 g, 4.26 mmol, yield: quantitative) of Compound 16.

LC/MS (ESI): m/z=369, RT=1.52 min, LC/MS measurement conditions 1

### Step 4 Synthesis of Compound 17

CD I (2.78 g, 17.2 mmol), Compound 16 (1.58 g, 4.29 mmol), and DMF (12.6 mL) were mixed. Diisopropylethylamine (3.00 mL, 17.2 mmol) was added to the reaction solution and the resultant solution was irradiated with microwave for 30 minutes while stirred at 110° C. The reaction solution was poured into ice, and the precipitates thus generated were filtered and washed with water. The residue thus obtained was dried under reduced pressure, thereby obtaining a crude product (649 mg, 1.51 mmol, yield 35%) of Compound 17.

LC/MS (ESI): m/z=395, RT=1.74 min, LC/MS measurement conditions 1

### Step 5 Synthesis of Compound (I-003)

6-Chloro-2-methyl-2H-indazole-5-amine (55.3 mg, 0.304 mmol), Compound 17 (100 mg, 0.254 mmol), and THF (1 mL) were mixed. The reaction solution was cooled in an ice bath, and LHMDS (0.761 mL, 0.761 mmol) was added thereto. The reaction solution was stirred in the ice bath for 40 minutes, and a saturated ammonium chloride aqueous solution was added thereto. The organic layer was extracted with ethyl acetate and concentrated under reduced pressure. The residue was purified with silica gel column chromatography (chloroform/methanol), thereby obtaining Compound (I-003) (80 mg, 0.145 mmol, yield 57.4%).

¹H-NMR (Methanol-d4) δ: 8.43 (d, J=1.0 Hz, 1H), 8.36 (d, J=2.0 Hz, 1H), 8.18 (s, 1H), 7.74 (s, 1H), 7.72 (br s, 1H), 7.48-7.35 (m, 3H), 5.32 (s, 2H), 4.20 (s, 3H), 2.42 (s, 3H).

LC/MS (ESI): m/z=528, RT=1.93 min, LC/MS measurement conditions 1

### Example 21

### Synthesis of Compound (I-005)

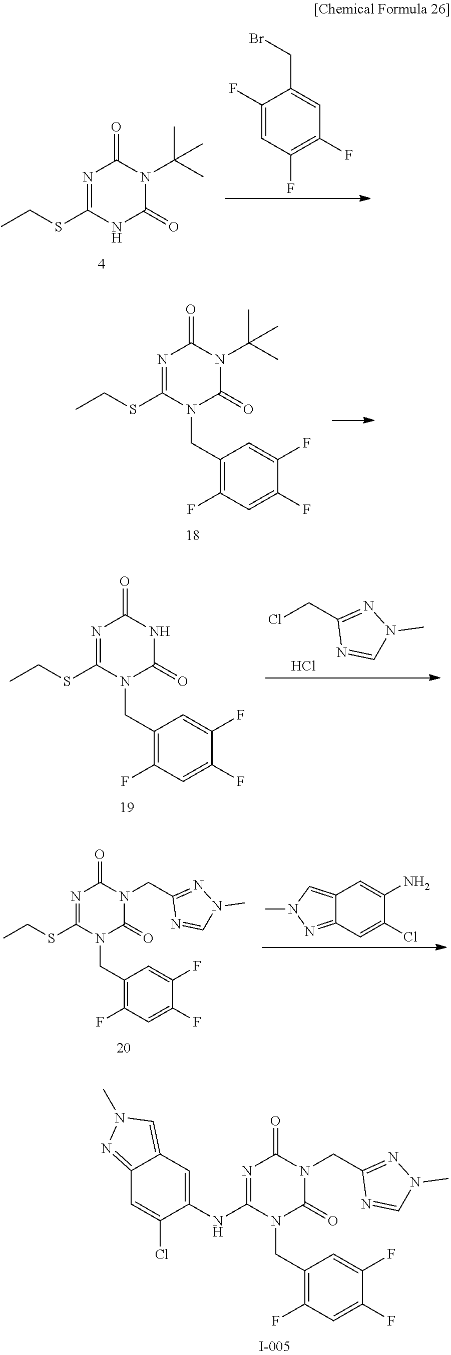

### Step 1 Synthesis of Compound 18

Compound 4 (926 mg, 4.04 mmol) (the synthesis method is referred to WO 2012020749 A, WO 2013089212 A, and WO 2014200078 A), acetonitrile (7.41 mL), potassium carbonate (726 mg, 5.25 mmol), and 2,4,5-trifluorobenzyl bromide (1000 mg, 4.44 mmol) were mixed. The reaction solution was stirred at 80° C. for 40 minutes, left to be cooled, and then diluted with ethyl acetate. The insoluble matters were filtered, and the filtrate was concentrated, thereby obtaining a crude product (1.51 g, 4.04 mmol, yield: quantitative) of Compound 18.

LC/MS (ESI): m/z=374, RT=2.54 min, LC/MS measurement conditions 1

### Step 2 Synthesis of Compound 19

Compound 18 (1.51 g, 4.04 mmol) and TFA (3.02 mL) were mixed. The reaction solution was stirred at room temperature for 4 hours and left to stand still overnight. TFA was distilled under reduced pressure, and the residue was azeotropically distilled by adding toluene thereto. The residue was suspended in isopropyl ether and then collected by filtration, thereby obtaining Compound 19 (1.22 g, 3.84 mmol, yield 95%).

LC/MS (ESI): m/z=318, RT=1.68 min, LC/MS measurement conditions 1

### Step 3 Synthesis of Compound 20

Compound 19 (200 mg, 0.63 mmol), DMF (1.8 mL), potassium carbonate (261 mg, 1.89 mmol), and 3-(chloromethyl)-1-methyl-1H-1,2,4-triazole hydrochloride (159 mg, 0.946 mmol) were mixed. The reaction solution was stirred at 60° C. for 2 hours, and a saturated ammonium chloride aqueous solution was added thereto. The aqueous layer was extracted with ethyl acetate, and the organic layer was washed with saturated saline. The organic layer was dried with magnesium sulfate, filtered, and concentrated. The residue was suspended in a mixed solvent of isopropyl ether, hexane, ethyl acetate, and chloroform and collected by filtration. The residue, DMF (1.8 mL), potassium carbonate (261 mg, 1.89 mmol), and 3-(chloromethyl)-1-methyl-1H-1,2,4-triazole hydrochloride (159 mg, 0.946 mmol) were mixed. The reaction solution was stirred at 60° C. for 6 hours, and a saturated ammonium chloride aqueous solution was added thereto. The aqueous layer was extracted with ethyl acetate, and the organic layer was washed with saturated saline. The organic layer was dried with magnesium sulfate, filtered, and concentrated. The residue was suspended in a mixed solvent of isopropyl ether, hexane, ethyl acetate, and chloroform and collected by filtration, thereby obtaining Compound 20 (116 mg, 0.281 mmol, yield 45%).

LC/MS (ESI): m/z=413, RT=1.84 min, LC/MS measurement conditions: 1

### Step 4 Synthesis of Compound (I-005)

Compound 20 (115 mg, 0.279 mmol), THE (2.30 mL), and 6-chloro-2-methyl-2H-indazole-5-amine (60.8 mg, 0.335 mmol) were mixed. LHMDS (558 μL, 0.558 mmol) was added dropwise at 0° C. to the reaction solution. The reaction solution was stirred at 0° C. for 2.5 hours and stirred at room temperature for 40 minutes, and a saturated ammonium chloride aqueous solution was added thereto. The aqueous layer was extracted with chloroform, and the organic layer was concentrated. The residue was purified with silica gel column chromatography (chloroform/methanol), thereby obtaining Compound (I-005) (61.8 mg, 0.116 mmol, yield 42%).

¹H-NMR (CDCl₃) δ: 7.96 (s, 1H), 7.82 (d, J=2.5 Hz, 2H), 7.48 (br s, 1H), 7.45-7.37 (m, 1H), 7.08 (s, 1H), 6.97-6.88 (m, 1H), 5.35 (s, 2H), 5.17 (s, 2H), 4.21 (s, 3H), 3.89 (s, 3H).

LC/MS (ESI): m/z=532, RT=1.70 min, LC/MS measurement conditions 1

The following compounds were synthesized according to the above-described general synthesis method and the methods described in Examples. The structure and physical properties (LC/MS data) are shown in the following tables.

TABLE 1

| | | LC/MS | Retention | |
|---|---|---|---|---|
| Compound | | measurement | time | |
| No. | Structure | condition | (min) | m/z |
| I-001 | 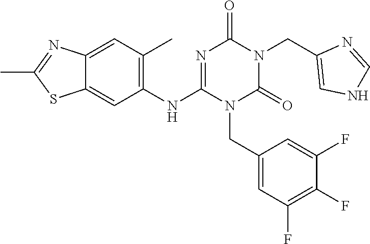 [SMILES: Cc1nc2cc(C)c(Nc3nc(=O)n(Cc4c[nH]cn4)c(=O)n3Cc3cc(F)c(F)c(F)c3)cc2s1] | 1 | 1.74 | 514.05 |
| I-002 | 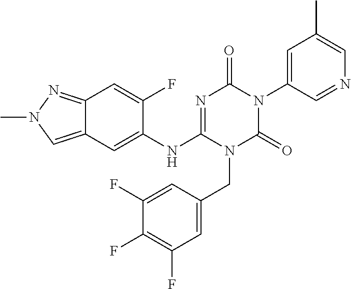 [SMILES: Cc1cncc(-n2c(=O)nc(Nc3cc4cn(C)nc4cc3F)n(Cc3cc(F)c(F)c(F)c3)c2=O)c1] | 1 | 1.80 | 512 |
| I-003 | 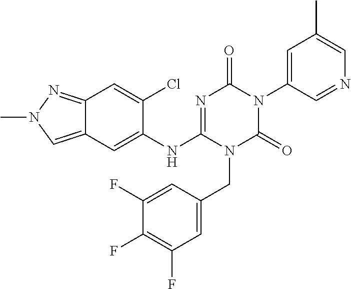 [SMILES: Cc1cncc(-n2c(=O)nc(Nc3cc4cn(C)nc4cc3Cl)n(Cc3cc(F)c(F)c(F)c3)c2=O)c1] | 1 | 1.93 | 528 |
| I-004 | 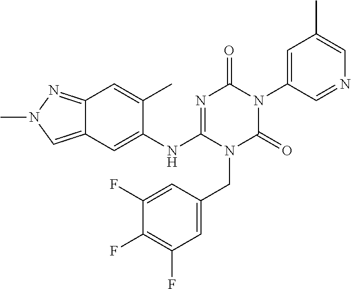 [SMILES: Cc1cncc(-n2c(=O)nc(Nc3cc4cn(C)nc4cc3C)n(Cc3cc(F)c(F)c(F)c3)c2=O)c1] | 1 | 1.68 | 508 |

TABLE 2

| | | LC/MS | Retention | |
|---|---|---|---|---|
| Compound | | measurement | time | |
| No. | Structure | condition | (min) | m/z |
| I-005 | 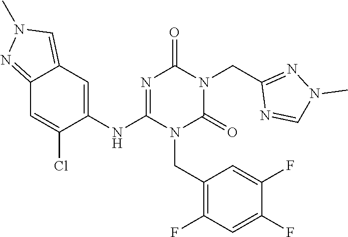 | 1 | 1.70 | 532 |
| I-006 | 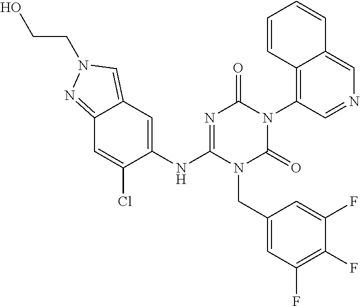 [SMILES: O=c1nc(Nc2cc3cn(CCO)nc3cc2Cl)n(Cc2cc(F)c(F)c(F)c2)c(=O)n1-c1cncc2ccccc12] | 2 | 1.82 | 593.95 |
| I-007 | 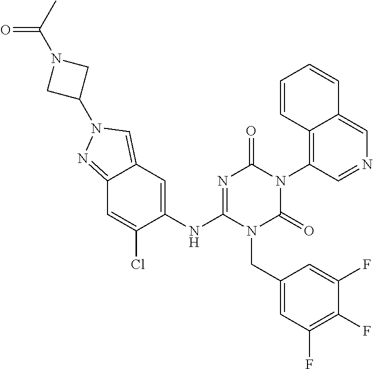 [SMILES: CC(=O)N1CC(n2cc3cc(Nc4nc(=O)n(-c5cncc6ccccc56)c(=O)n4Cc4cc(F)c(F)c(F)c4)c(Cl)cc3n2)C1] | 1 | 1.93 | 647.15 |
| I-008 | 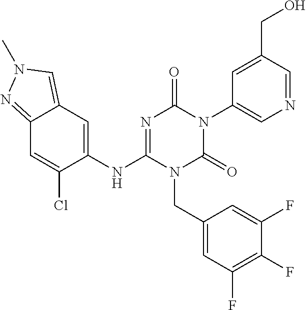 [SMILES: Cn1cc2cc(Nc3nc(=O)n(-c4cncc(CO)c4)c(=O)n3Cc3cc(F)c(F)c(F)c3)c(Cl)cc2n1] | 1 | 1.54 | 510.2 |

TABLE 3

| | | LC/MS | Retention | |
|---|---|---|---|---|
| Compound | | measurement | time | |
| No. | Structure | condition | (min) | m/z |
| I-009 | 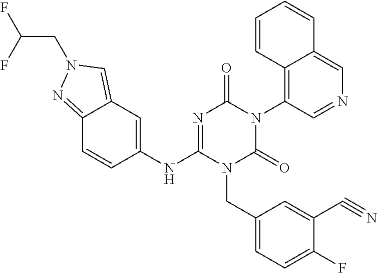 [SMILES: N#Cc1cc(Cn2c(Nc3ccc4nn(CC(F)F)cc4c3)nc(=O)n(-c3cncc4ccccc34)c2=O)ccc1F] | 1 | 1.83 | 569.2 |
| I-010 | 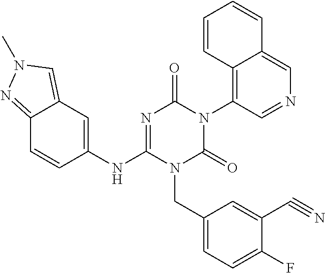 [SMILES: Cn1cc2cc(Nc3nc(=O)n(-c4cncc5ccccc45)c(=O)n3Cc3ccc(F)c(C#N)c3)ccc2n1] | 1 | 1.68 | 519.2 |
| I-011 | 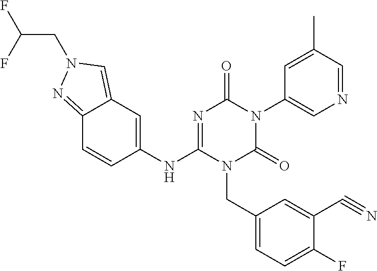 [SMILES: Cc1cncc(-n2c(=O)nc(Nc3ccc4nn(CC(F)F)cc4c3)n(Cc3ccc(F)c(C#N)c3)c2=O)c1] | 1 | 1.68 | 533 |
| I-012 | 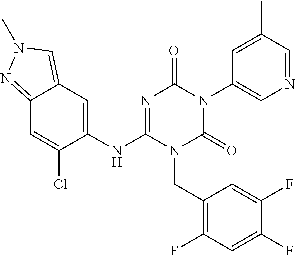 [SMILES: Cc1cncc(-n2c(=O)nc(Nc3cc4cn(C)nc4cc3Cl)n(Cc3cc(F)c(F)cc3F)c2=O)c1] | 1 | 1.85 | 528 |

TABLE 4

| | | LC/MS | Retention | |
|---|---|---|---|---|
| Compound | | measurement | time | |
| No. | Structure | condition | (min) | m/z |
| I-013 | 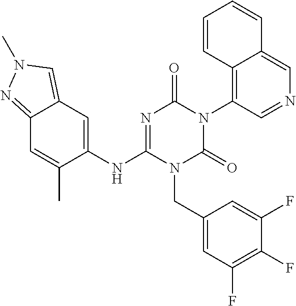 [SMILES: Cc1cc2nn(C)cc2cc1Nc1nc(=O)n(-c2cncc3ccccc23)c(=O)n1Cc1cc(F)c(F)c(F)c1] | 1 | 1.81 | 544 |
| I-014 | 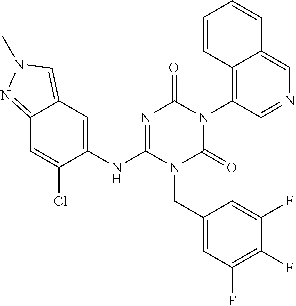 [SMILES: Cn1cc2cc(Nc3nc(=O)n(-c4cncc5ccccc45)c(=O)n3Cc3cc(F)c(F)c(F)c3)c(Cl)cc2n1] | 2 | 1.96 | 564 |
| I-015 | 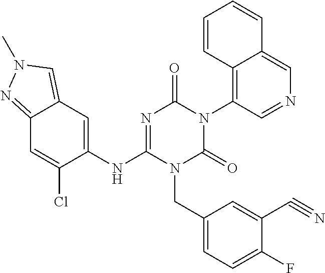 [SMILES: Cn1cc2cc(Nc3nc(=O)n(-c4cncc5ccccc45)c(=O)n3Cc3ccc(F)c(C#N)c3)c(Cl)cc2n1] | 2 | 1.76 | 553 |
| I-016 | 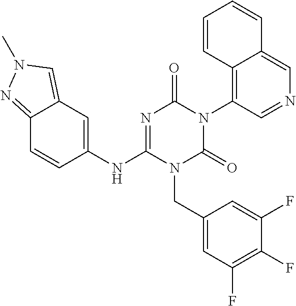 [SMILES: Cn1cc2cc(Nc3nc(=O)n(-c4cncc5ccccc45)c(=O)n3Cc3cc(F)c(F)c(F)c3)ccc2n1] | 2 | 1.70 | 530 |

TABLE 5

| | | LC/MS | Retention | |
|---|---|---|---|---|
| Compound | | measurement | time | |
| No. | Structure | condition | (min) | m/z |
| I-017 | 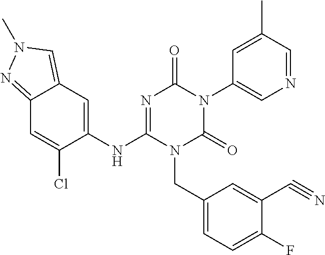 [SMILES: Cc1cncc(-n2c(=O)nc(Nc3cc4cn(C)nc4cc3Cl)n(Cc3ccc(F)c(C#N)c3)c2=O)c1] | 2 | 1.64 | 517 |
| I-018 | 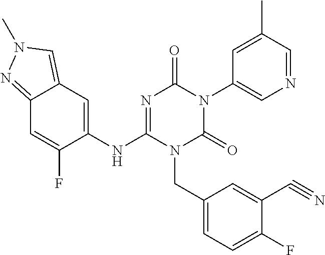 [SMILES: Cc1cncc(-n2c(=O)nc(Nc3cc4cn(C)nc4cc3F)n(Cc3ccc(F)c(C#N)c3)c2=O)c1] | 2 | 1.54 | 501 |
| I-019 | 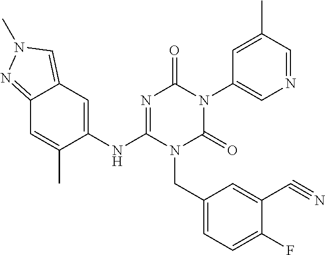 [SMILES: Cc1cncc(-n2c(=O)nc(Nc3cc4cn(C)nc4cc3C)n(Cc3ccc(F)c(C#N)c3)c2=O)c1] | 2 | 1.49 | 497 |
| I-020 | 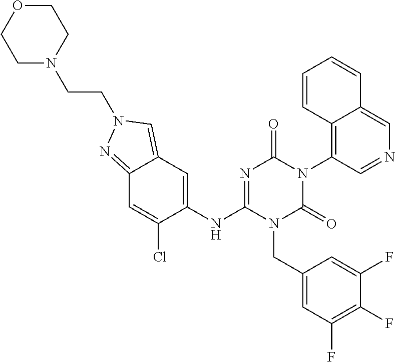 [SMILES: O=c1nc(Nc2cc3cn(CCN4CCOCC4)nc3cc2Cl)n(Cc2cc(F)c(F)c(F)c2)c(=O)n1-c1cncc2ccccc12] | 1 | 1.68 | 663.25 |

TABLE 6

| | | LC/MS | Retention | |
|---|---|---|---|---|
| Compound | | measurement | time | |
| No. | Structure | condition | (min) | m/z |
| I-021 | 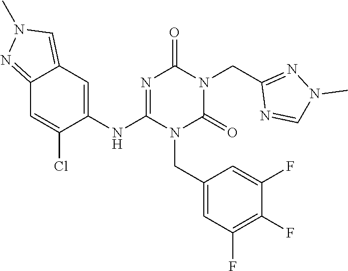 | 2 | 1.80 | 532 |
| I-022 | 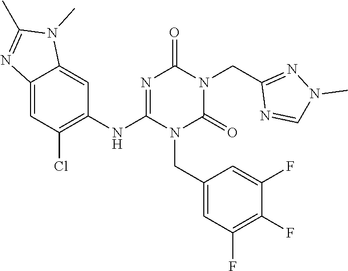 | 2 | 1.49 | 546 |
| I-023 | 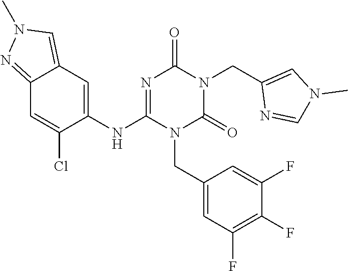 | 2 | 1.31 | 531 |
| I-024 | 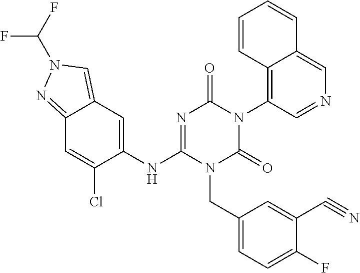 [SMILES: N#Cc1cc(Cn2c(Nc3cc4cn(C(F)F)nc4cc3Cl)nc(=O)n(-c3cncc4ccccc34)c2=O)ccc1F] | 2 | 2.04 | 589 |

TABLE 7

| | | LC/MS | Retention | |
|---|---|---|---|---|
| Compound | | measurement | time | |
| No. | Structure | condition | (min) | m/z |
| I-025 | 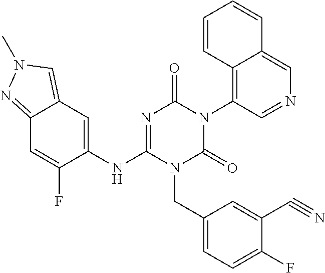 [SMILES: Cn1cc2cc(Nc3nc(=O)n(-c4cncc5ccccc45)c(=O)n3Cc3ccc(F)c(C#N)c3)c(F)cc2n1] | 2 | 1.76 | 537 |
| I-026 | 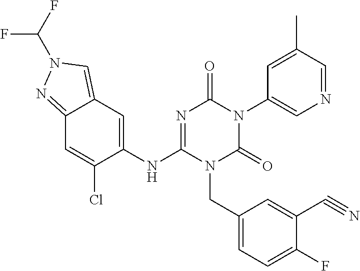 [SMILES: Cc1cncc(-n2c(=O)nc(Nc3cc4cn(C(F)F)nc4cc3Cl)n(Cc3ccc(F)c(C#N)c3)c2=O)c1] | 2 | 2.05 | 553 |
| I-027 | 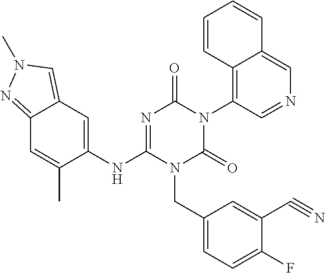 [SMILES: Cc1cc2nn(C)cc2cc1Nc1nc(=O)n(-c2cncc3ccccc23)c(=O)n1Cc1ccc(F)c(C#N)c1] | 2 | 1.61 | 533 |
| I-028 | 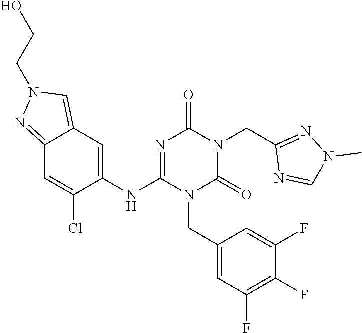 [SMILES: Cn1cnc(Cn2c(=O)nc(Nc3cc4cn(CCO)nc4cc3Cl)n(Cc3cc(F)c(F)c(F)c3)c2=O)n1] | 2 | 1.69 | 562 |

TABLE 8

| | | LC/MS | Retention | |
|---|---|---|---|---|
| Compound | | measurement | time | |
| No. | Structure | condition | (min) | m/z |
| I-029 | 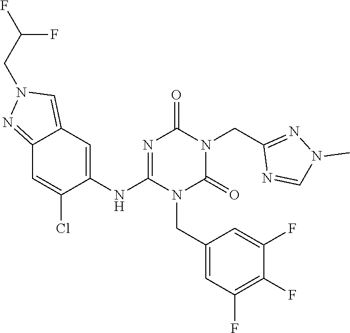 [SMILES: Cn1cnc(Cn2c(=O)nc(Nc3cc4cn(CC(F)F)nc4cc3Cl)n(Cc3cc(F)c(F)c(F)c3)c2=O)n1] | 2 | 1.98 | 582 |
| I-030 | 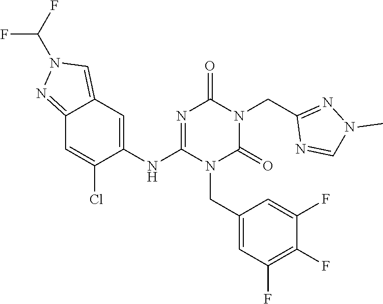 [SMILES: Cn1cnc(Cn2c(=O)nc(Nc3cc4cn(C(F)F)nc4cc3Cl)n(Cc3cc(F)c(F)c(F)c3)c2=O)n1] | 2 | 2.00 | 568 |
| I-031 | 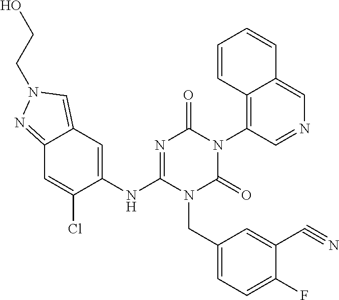 [SMILES: N#Cc1cc(Cn2c(Nc3cc4cn(CCO)nc4cc3Cl)nc(=O)n(-c3cncc4ccccc34)c2=O)ccc1F] | 2 | 1.74 | 583 |
| I-032 | 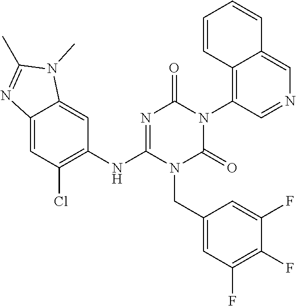 [SMILES: Cc1nc2cc(Cl)c(Nc3nc(=O)n(-c4cncc5ccccc45)c(=O)n3Cc3cc(F)c(F)c(F)c3)cc2n1C] | 1 | 1.71 | 578.2 |

TABLE 9

| | | LC/MS | Retention | |
|---|---|---|---|---|
| Compound | | measurement | time | |
| No. | Structure | condition | (min) | m/z |
| I-033 | 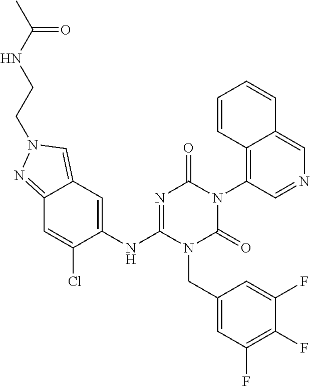 [SMILES: CC(=O)NCCn1cc2cc(Nc3nc(=O)n(-c4cncc5ccccc45)c(=O)n3Cc3cc(F)c(F)c(F)c3)c(Cl)cc2n1] | 1 | 1.94 | 635 |
| I-034 | 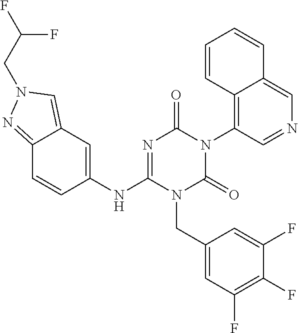 [SMILES: O=c1nc(Nc2ccc3nn(CC(F)F)cc3c2)n(Cc2cc(F)c(F)c(F)c2)c(=O)n1-c1cncc2ccccc12] | 2 | 1.87 | 579.95 |
| I-035 | 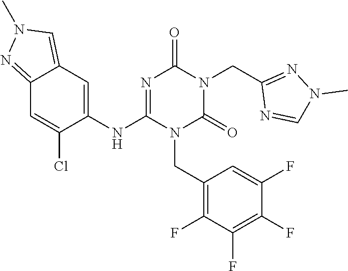 | 1 | 1.93 | 550 |

### Example 31

To 1170 mg of Compound (I-005), 278 mg (1.1 eq) of fumaric acid and 5.85 mL of ethyl acetate were added, and the resultant product was stirred at room temperature for 45 minutes. The solid was collected by filtration and dried, there by obtaining fumaric acid cocrystal Form I crystal (1369.4 mg, 94.6%) of the compound represented by Formula (I-B).

The result of the single crystal structural analysis of fumaric acid cocrystal Form I of the compound represented by Formula (I-B) is shown below.

R¹ (I>2.00s(I)) was 0.0470, and it was confirmed that there is neither a lack of electronic density neither misplacing of atom from final difference Fourier.

Crystallographic data are shown in Table 10.

TABLE 10

| Space Group | P-1 |
|---|---|
| a (Å) | 8.4374(2) |
| b (Å) | 11.6780(3) |
| c (Å) | 15.1612(4) |
| α (°) | 83.827(2) |
| β (°) | 78.868(2) |
| γ (°) | 77.147(2) |
| Volume (Å³) | 1425.77(6) |
| Z | 2 |
| Density (calculated value) (g/cm³) | 1.509 |
| Measured temperature (K) | 298 |

Herein, Volume means the unit lattice volume, and Z means the number of molecules in the unit lattice.

The structure of the fumaric acid cocrystal Form I of the compound represented by Formula (I-B) in the asymmetric unit is shown in FIG. 2.

One molecule of each of the compound represented by Formula (I-B) and fumaric acid was present in the asymmetric unit. The ionic chemical interaction was not confirmed, and the structure was confirmed to be a cocrystal at a molar ratio of 1:1.

The bond length of N10-C9 was about 1.26 Å, and the bond length of N16-C9 was about 1.37 Å. From this bond length, the compound represented by Formula (I-B) of the fumaric acid cocrystal Form I was identified to have an imino structure:

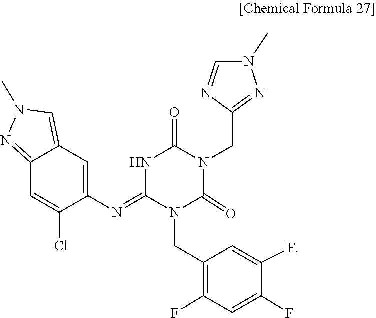 [SMILES: Cn1cnc(Cn2c(=O)[nH]/c(=N\c3cc4cn(C)nc4cc3Cl)n(Cc3cc(F)c(F)cc3F)c2=O)n1]

Furthermore, the result of the X-ray powder diffraction of fumaric acid cocrystal Form I crystal of the compound represented by Formula (I-B) is shown.

In the X-ray powder diffraction pattern, the peaks were observed at the diffraction angle (2θ): 7.8±θ.2°, 9.5±0.2°, 10.1±0.2°, 10.9±0.2°, 13.8±0.2°, 14.7±0.2°, 18.6±0.2°, 22.6±0.2°, 23.5±0.2°, and 24.6±0.2°.

In the above-described X-ray powder diffraction peaks, the peaks of the diffraction angle (2θ): 9.5±0.2°, 10.9±0.2°, 18.6±0.2°, 23.5±0.2°, and 24.6±0.2° are particularly characteristic as the fumaric acid cocrystal Form I crystal of the compound represented by Formula (I-B).

Hereinafter, biological test examples relevant to the medicament of the present invention are described.

The compound represented by (A) in the medicament of the present invention may have coronavirus 3CL protease inhibitory action and may inhibit coronavirus 3CL protease.

Specifically, in the evaluation method described below, IC50 is preferably 50 μM or less, more preferably 1 μM or less, and even more preferably 100 nM or less.

### Test Example 1: Cytopathic Effect (CPE) Suppression Effect Confirmation Test Using Vero E6 Cells Expressing Human TMPRSS2 (Vero E6/TMPRSS2 Cells)

### <Operation Procedure>

### Diluting and Dispensing of Test Sample

The test sample is preliminarily diluted with DMSO to an appropriate concentration, and a 2- to 5-fold serial dilution series is prepared and then dispensed into a 384-well plate.

### Diluting and Dispensing of Cells and SARS-CoV-2

VeroE6/TMPRSS2 cells (JCRB1819, 5×10³ cells/well) and SARS-CoV-2 (100 TCID₅₀/well) are mixed in a culture medium (MEM, 2% FBS, penicillin-streptomycin), dispensed into a well containing the test sample, and then cultured in a CO₂ incubator for 3 days.

### Dispensing and Measuring of Luminescence Signal of CellTiter-Glo (Registered Trademark) 2.0

After returning the plate cultured for 3 days to room temperature, CellTiter-Glo (registered trademark) 2.0 is dispensed into each well and mixed with a plate mixer. After a certain time interval, the luminescence signal (Lum) is measured with a plate reader.

### <Calculation of Each Measurement Item>

### Calculation of 50% SARS-CoV-2 Infected Cell Death Inhibitory Concentration (EC₅₀)

When x is taken as the logarithmic value of the compound concentration and y as % Efficacy, the inhibition curve is approximated by the following Logistic regression equation, and the value of x when substituting 50(%) for y is calculated as EC₅₀.

y=min+(max−min)/{1+(X50/x)\hat{ }Hill}

% Efficacy={(Sample−virus control)/(cell control−virus control)}*100%
cell control: the average of Lum of cell control wells
virus control: the average of Lum of virus control wells
min: lower limit of y-axis, max: upper limit of y-axis, X50: x-coordinate of inflection point, Hill: slope of curve at midpoint between min and max

The compound represented by (A) in the medicament of the present invention was tested essentially as described above. Results are shown below.

Note that, EC₅₀ value is set as “A” for less than 1 μM and “B” for 1 μM or more and less than 10 μM.
Compound I-003: 0.177 μM
Compound I-005: 0.328 μM
Compound I-006: 0.747 μM
Compound I-010: 0.306 μM
Compound I-012: 0.131 μM
Compound I-017: 0.0960 μM
Compound I-023: 1.03 μM
Compound I-035: 0.395 μM

TABLE 11

| Compound No. | EC50 |
|---|---|
| I-002 | A |
| I-004 | B |
| I-007 | C |
| I-009 | A |
| I-011 | A |
| I-013 | B |
| I-014 | A |
| I-015 | A |
| I-016 | A |
| I-018 | A |
| I-019 | A |
| I-020 | B |
| I-021 | A |
| I-022 | B |
| I-024 | A |
| I-025 | A |
| I-026 | A |
| I-027 | A |
| I-028 | B |
| I-029 | A |
| I-030 | B |
| I-031 | B |
| I-032 | B |
| I-033 | B |
| I-034 | A |

### Test Example 2: Inhibitory Activity Test Against SARS-CoV-2 3CL Protease

### <Material>

### Commercially Available Recombinant SARS-CoV-2 3CL Protease

### Commercially Available Substrate Peptide

Dabcyl-Lys-Thr-Ser-Ala-Val-Leu-Gln-Ser-Gly-Phe-Arg-Lys-Met-Glu(Edans)-NH2 (SEQ ID NO: 1)

### Internal Standard Peptide

Dabeyl-Lys-Thr-Ser-Ala-Val-Leu(13C6,15N)-Gln (SEQ ID NO: 2)

Dabcyl-Lys-Thr-Ser-Ala-Val-Leu(13C6,15N)-Gln can be synthesized with reference to the literature (Atherton, E.; Sheppard, R. C., “In Solid Phase Peptide Synthesis, A Practical Approach”, IRL Press at Oxford University Pres, 1989. and Bioorg. Med. Chem., Volume 5, Issue 9, 1997, pp. 1883-1891, etc.). An example will be described below.

H-Lys-Thr-Ser-Ala-Val-Leu(13C6,15N)-Glu(resin)-OaOtBu (the Lys side chain is Boc-protected, the Thr side chain is protected with a tert-butyl group, the Ser side chain is protected with a tert-butyl group, the C-terminal OH of Glu is protected with a tert-butyl group, and the carboxylic acid of the Glu side chain is condensed into the resin) is synthesized using Rink amide resin by Fmoc solid-phase synthesis. Modification of the N-terminus Dabcyl group condenses 4-dimethylaminoazobenzene-4′-carboxylic acid (Dabcyl-OH) on resins using EDC/HOBT. Final deprotection and excision from the resins are performed by treatment with TFA/EDT=95:5. Thereafter, purification is performed by reverse phase HPLC.

### RapidFire Cartridge C4 Type a

### <Operation Procedure>

### Preparation of Assay Buffer

In this test, an assay buffer consisting of 20 mM Tris-HCl, 100 mM sodium chloride, 1 mM EDTA, 10 mM DTT, and 0.01% BSA is used. For compounds with an IC₅₀ value of 10 nM or less, an assay buffer consisting of 20 mM Tris-HCl, 1 mM EDTA, 10 mM DTT, and 0.01% BSA is used.

### Diluting and Dispensing of Test Sample

The test sample is preliminarily diluted with DMSO to an appropriate concentration, and a 2- to 5-fold serial dilution series is prepared and then dispensed into a 384-well plate.

### Addition of Enzyme and Substrate and Enzymatic Reaction

To the prepared compound plate, 8 μM of substrate and 6 or 0.6 nM of enzyme solution are added and incubation is performed for 3 to 5 hours at room temperature. Thereafter, a reaction stop solution (0.067 μM Internal Standard, 0.1% formic acid, 10 or 25% acetonitrile) is added to stop the enzymatic reaction.

### Measurement of Reaction Product

The plate in which the reaction has been completed is measured using RapidFire System 360 and mass spectrometer (Agilent Technologies, Inc., 6550 iFunnel Q-TOF), or Rapid Fire System 365 and mass spectrometer (Agilent Technologies, Inc., 6495C Triple Quadrupole). As the mobile phase at the time of measurement, A solution (75% isopropanol, 15% acetonitrile, 5 mM ammonium formate) and B solution (0.01% trifluoroacetic acid, 0.09% formic acid) are used.

Reaction products detected by the mass spectrometer are calculated using RapidFire Integrator or a program capable of performing equivalent analysis and are taken as Product area value. Furthermore, Internal Standard detected at the same time is also calculated and taken as Internal Standard area value.

### <Calculation of Each Measurement Item>

### Calculation of P/IS

The area values obtained in the previous section is calculated by the following equation to calculate P/IS.

P/IS=Product area value/Internal Standard area value

### Calculation of 50% SARS-CoV-2 3CL Protease Inhibitory Concentration (IC₅₀)

When x is taken as the logarithmic value of the compound concentration and y as % Inhibition, the inhibition curve is approximated by the following Logistic regression equation, and the value of x when substituting 50(%) for y is calculated as IC₅₀.

y=min+(max−min)/{1+(X50/x)\hat{ }Hill}

% Inhibition={1−(Sample−Control(−))/Control(+)−Control(−))}*100
Control(−): the average of P/IS of enzyme inhibited condition wells
Control(+): the average of P/IS of DMSO control wells
min: lower limit of y-axis, max: upper limit of y-axis, X50: x-coordinate of inflection point, Hill: slope of curve at midpoint between min and max

The compound represented by (A) in the medicament of the present invention was tested essentially as described above. Results are shown below.

Note that, IC₅₀ value is set as “A” for less than 0.1 μM, “B” for 0.1 μM or more and less than 1 μM, and “C” for 1 μM or more and less than 10 μM.
Compound I-003: 0.014 μM
Compound I-005: 0.010 μM
Compound I-006: 0.0058 μM
Compound I-010: 0.0054 μM
Compound I-012: 0.0091 μM
Compound I-017: 0.0034 μM
Compound I-023: 0.0063 μM
Compound I-035: 0.0098 μM

TABLE 12

| Compound No. | IC50 |
|---|---|
| I-001 | C |
| I-002 | A |
| I-004 | A |
| I-007 | A |
| I-008 | B |
| I-009 | A |
| I-011 | A |
| I-013 | A |
| I-014 | A |
| I-015 | A |
| I-016 | A |
| I-018 | A |
| I-019 | A |
| I-020 | A |
| I-021 | A |
| I-022 | A |
| I-024 | A |
| I-025 | A |
| I-026 | A |
| I-027 | A |
| I-028 | A |
| I-029 | A |
| I-030 | A |
| I-031 | A |
| I-032 | A |
| I-033 | A |
| I-034 | A |

### Test Example 3: Combination Effect Confirmation Test

### <Operation Procedure>

### Diluting and Dispensing of Test Sample

Each test sample is diluted with DMSO and a culture medium (MEM, 2% FBS, penicillin-streptomycin) and serial dilution series is prepared in a 96-well plate.

### Diluting and Dispensing of Cells and SARS-CoV-2

VeroE6/TMPRSS2 cells (JCRB1819, 1.5×10⁴ cells/well) and SARS-CoV-2 (1000 TCID₅₀/well) are mixed in a culture medium (MEM, 2% FBS, penicillin-streptomycin), dispensed into a well containing the test sample, and then cultured in a CO₂ incubator for 3 days.

### Dispensing and Measuring of Luminescence Signal of CellTiter-Glo (Registered Trademark) 2.0

After returning the plate cultured for 3 days to room temperature, CellTiter-Glo (registered trademark) 2.0 is dispensed into each well and mixed with a plate mixer. After a certain time interval, the luminescence signal (Lum) is measured with a plate reader.

### <Calculation of Each Measurement Item>

The combination index (CI) can be calculated with reference to the literature of Chou T. C., et al. (Advances in Enzyme Regulation, 1984, Volume 22, Issue C, p. 27-55) and the like.

### Calculation of 50% SARS-CoV-2 Infected Cell Death Inhibitory Concentration (EC₅₀)

While cells not infected with viruses are regarded as 100% inhibition and cells cultured under the condition not containing a test sample after virus infection are regarded as 0% inhibition, the SARS-CoV-2-infected cell death inhibition ratio of the test sample is calculated, and then EC₅₀ is calculated using XL fit 5.3.1.3.

### Calculation of Fractional Inhibitory Concentration (FIC)

$$FIC ( A ) = ( {D}_{A / A + B} ) / {D}_{A}$$

$$FIC ( B ) = ( {D}_{B / A + B} ) / {D}_{B}$$

D_{A}: EC₅₀ of test substance A alone
D_{B}: EC₅₀ of test substance B alone
D_{A/A+B}: concentration of test substance A giving 50% inhibition of SARS-CoV-2 infected cell death when using test substances A and B in combination
D_{B/A+B}: concentration of test substance B giving 50% inhibition of SARS-CoV-2 infected cell death when using test substances A and B in combination

### Calculation of Combination Index (CI)

The CI value when using the test substances A and B in combination at a ratio corresponding to the ratio of the EC₅₀ value of each single agent is calculated.

$$C I = FIC ( A ) + FIC ( B ) + FIC ( A ) \times FIC ( B )$$

### <Determination of Combination Effect>

According to the paper of Naruto Taira, et al. (Acta Med. Okayama, 2006 vol. 60, p 25-34), combination effects are analyzed as follows: synergy when CI<0.8, additive when 0.8<CI<1.2, and antagonism when 1.2<CI.

The test was conducted essentially as described above. Results are shown in the following table.

The EC₅₀ values of respective single agents are shown in Tables 13 to 16.

TABLE 13

| | | | Fumaric acid cocrystal |
|---|---|---|---|
| | | | Form I of the |
| | | | compound represented |
| | | | by formula |
| | (I-005) | (I-003) | (I-B) |
| hCoV-19/Japan/ | 0.328 [μM] | 0.177 [μM] | 0.37 [μM] |
| TY/WK-521/2020 | | | |
| hCoV-19/Japan/ | — | — | 0.50 [μM] |
| TY7-501/2021 | | | |
| hCoV-19/Japan/ | — | — | 0.41 [μM] |
| TY11-927-P1/2021 | | | |

TABLE 14

| | EIDD-1931 | | Remdesivir | PF-07321332 | Camostatmesylate |
|---|---|---|---|---|---|
| hCoV-19/Japan/TY/WK-521/2020 | 0.69 | [μM] | 2.5 [μM] | 7.1 [μM] | 1.3 [μM] |
| hCoV-19/Japan/TY7-501/2021 | 1.5 | [μM] | 2.3 [μM] | — | — |

| | | | | |
|---|---|---|---|---|
| hCoV-19/Japan/TY11-927-P1/2021 | — | 2.0 [μM] | — | — |

TABLE 15

| | Casirivimab | Imdevimab | Casirivimab and imdevimab |
|---|---|---|---|
| hCoV-19/Japan/TY/WK-521/2020 | 0.077 [μg/mL] | 0.047 [μg/mL] | 0.046 [μg/mL] |
| hCoV-19/Japan/TY7-501/2021 | — | — | 0.034 [μg/mL] |
| hCoV-19/Japan/TY11-927-P1/2021 | — | — | 0.033 [μg/mL] |

TABLE 16

| | | | Tixagevimab |
|---|---|---|---|
| Virus | Sotrovimab | Bebtelovimab | and cilgavimab |
| hCoV-19/Japan/ | 1.06(μg/mL) | 0.0231(μg/mL) | 0.223(μg/mL) |
| TY11-927-P1/2021 | | | |

The CI values when using Compound (I-005) (free form) and the COVID-19 exacerbation suppressant in combination at a ratio corresponding to the ratio of the EC₅₀ value of each single agent are shown in Table 17.

TABLE 17

| | CI value | | | | | |
|---|---|---|---|---|---|---|
| | | | | | | (I-005) + |
| | (I-005) + | (I-005) + | (I-005) + | (I-005) + | (I-005) + | camostat |
| Virus | EIDD-1931 | remdesivir | casirivimab | imdevimab | PF-07321332 | mesylate |
| hCoV-19/Japan/TY/ | 1.17 | 1.01 | 0.747 | 0.696 | 1.19 | 1.18 |
| WK-521/2020 | | | | | | |

The CI values when using Compound (I-003) (free form) and the COVID-19 exacerbation suppressant in combination at a ratio corresponding to the ratio of the EC₅₀ value of each single agent are shown in Table 18.

TABLE 18

CI value

| | (I-003) + | (I-003) + | (I-003) + |
|---|---|---|---|
| Virus | EIDD-1931 | remdesivir | PF-07321332 |
| hCoV-19/Japan/ | 1.12 | 0.902 | 1.17 |
| TY/WK-521/2020 | | | |

The CI values when using the fumaric acid cocrystal Form I of the compound represented by Formula (I-B) and the COVID-19 exacerbation suppressant in combination at a ratio corresponding to the ratio of the EC₅₀ value of each single agent are shown in Table 19 and Table 20.

TABLE 19

| | CI value | | |
|---|---|---|---|
| | Fumaric acid cocrystal | Fumaric acid cocrystal | Fumaric acid cocrystal Form I of |
| | Form I of the compound | Form I of the compound | the compound represented by |
| | represented by formula | represented by formula | formula (I-B) + casirivimab and |
| Virus | (I-B) + EIDD-1931 | (I-B) + remdesivir | imdevimab |
| hCoV-19/Japan/TY/WK-521/2020 | 1.12 | 1.10 | 0.768 |
| hCoV-19/Japan/TY7-501/2021 | 1.16 | 0.978 | 0.778 |
| hCoV-19/Japan/TY11-927-P1/2021 | — | 1.11 | 0.913 |
| | | 1.06 | 0.742 |
| | | 1.03 | 0.730 |

TABLE 20

| | CI value | | |
|---|---|---|---|
| | Fumaric acid cocrystal | Fumaric acid cocrystal | Fumaric acid cocrystal Form I of |
| | Form I of the compound | Form I of the compound | the compound represented by |
| | represented by formula | represented by formula | formula (I-B) + tixagevimab and |
| Virus | (I-B) + sotrovimab | (I-B) + bebtelovimab | cilgavimab |
| hCoV-19/Japan/TY11-927-P1/2021 | 0.874 | 1.04 | 1.18 |
| | 0.971 | 0.938 | 1.19 |
| | 0.828 | 0.992 | 1.04 |

From the above results, it has been found that combination administration of Compound (I-005) and an anti-SARS-CoV-2 monoclonal antibody (Casirivimab and Imdevimab) and combination administration of the fumaric acid cocrystal Form I of the compound represented by Formula (I-B) and an anti-SARS-CoV-2 monoclonal antibody (Casirivimab and Imdevimab) exhibit additive to synergistic SARS-CoV-2 proliferation inhibitory effect, as compared with each single agent administration. Based on this fact, it has been found that, by combining the compound represented by Formula (I) or a pharmaceutically acceptable salt thereof with an anti-SARS-CoV-2 monoclonal antibody, additive to synergistic SARS-CoV-2 proliferation inhibitory effect is exhibited.

Furthermore, combination administration of Compound (I-005) and an RNA-dependent RNA polymerase inhibitor (EIDD-1931 and Remdesivir), combination administration of Compound (I-003) and an RNA-dependent RNA polymerase inhibitor (EIDD-1931 and Remdesivir), combination administration of the fumaric acid cocrystal Form I of the compound represented by Formula (I-B) and an RNA-dependent RNA polymerase inhibitor (EIDD-1931 and Remdesivir), combination administration of Compound (I-005) and a 3CL protease inhibitor (PF-07321332), combination administration of Compound (I-003) and a 3CL protease inhibitor (PF-07321332), combination administration of Compound (I-005) and a TMPRSS2 inhibitor (Camostat), and combination administration of the fumaric acid cocrystal Form I of the compound represented by Formula (I-B) and an anti-SARS-CoV-2 monoclonal antibody (Sotrovimab, Bebtelovimab, Tixagevimab, and Cilgavimab) also exhibited additive SARS-CoV-2 proliferation inhibitory effect without exhibiting antagonistic effect, as compared with each single agent administration. Therefore, it has been found that, by combining the compound represented by Formula (I) or a pharmaceutically acceptable salt thereof with an RNA-dependent RNA polymerase inhibitor, a 3CL protease inhibitor, a TMPRSS2 inhibitor, and an anti-SARS-CoV-2 monoclonal antibody, additive SARS-CoV-2 proliferation inhibitory effect is exhibited.

The following formulation examples are merely examples and not intended to limit the scope of the invention.

The compound in the medicament or the enhancer of the present invention can be administered as a pharmaceutical composition in any conventional route, particularly, in an enteral route, for example, orally, for example, in the form of a tablet or a capsule, or parenterally, for example, in the form of an injection or a suspension, locally, for example, in the form of a lotion, a gelling agent, an ointment, or a cream, or in the intranasal form or suppository form. The medicament or the enhancer of the present invention in the free form or in the form of a pharmaceutically acceptable salt can be produced together with at least one kind of pharmaceutically acceptable carrier or diluent by a conventional method such as a mixing, granulating, or coating method. For example, as a composition for oral, tablets, granules, and capsules containing excipients, disintegrants, binders, lubricants, etc. and an active ingredient, etc. can be used. Furthermore, as a composition for injection, solutions or suspensions can be used, and sterilization can be carried out, or preservatives, stabilizing agents, buffer agents, and the like may be contained.

### INDUSTRIAL APPLICABILITY

It is conceivable that the medicament or the enhancer of the present invention is useful as a therapeutic agent for symptoms and/or diseases induced by infection with SARS-CoV-2 and a prophylactic agent for symptoms and/or diseases induced by infection with SARS-CoV-2.

### SEQUENCE LISTING

1. A medicament characterized by combining (A) a compound represented by Formula (I): 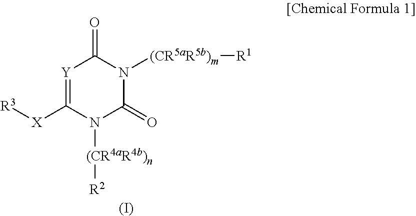
wherein Y is N;
R¹ is substituted or unsubstituted aromatic heterocyclyl;
R² is substituted or unsubstituted 6-membered aromatic carbocyclyl;
R³ is substituted or unsubstituted aromatic heterocyclyl;
—X— is —NH—;
m is 0 or 1;
R^{5a} is a hydrogen atom;
R^{5b} is a hydrogen atom;
n is 1;
R^{4a} is a hydrogen atom; and
R^{4b} is a hydrogen atom, or a pharmaceutically acceptable salt thereof; and
(B) a COVID-19 exacerbation suppressant (provided that, excluding the compound represented by Formula (I) or a pharmaceutically acceptable salt thereof).

2. The medicament according to claim 1, wherein R¹ is substituted or unsubstituted 5- to 6-membered aromatic heterocyclyl.

3. The medicament according to claim 1, wherein
R² is 6-membered aromatic carbocyclyl substituted with one, two, or three substituents selected from a substituent group G; and
the substituent group G described here is a group consisting of halogen, cyano, and unsubstituted alkyl.

4. The medicament according to claim 1, wherein R³ is substituted or unsubstituted 9- to 10-membered aromatic heterocyclyl.

5. The medicament according to claim 1, wherein
(A) is a compound represented by formula: 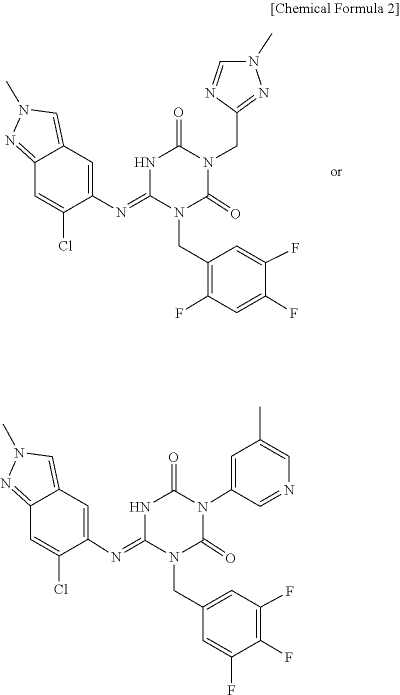 [SMILES: Cn1cnc(Cn2c(=O)[nH]/c(=N\c3cc4cn(C)nc4cc3Cl)n(Cc3cc(F)c(F)cc3F)c2=O)n1 ; Cc1cncc(-n2c(=O)[nH]/c(=N\c3cc4cn(C)nc4cc3Cl)n(Cc3cc(F)c(F)c(F)c3)c2=O)c1]
or a pharmaceutically acceptable salt thereof.

6. The medicament according to claim 1, wherein (B) is at least one selected from the group consisting of an anti-SARS-CoV-2 monoclonal antibody, an RNA-dependent RNA polymerase inhibitor, a 3CL protease inhibitor (provided that, excluding the compound represented by Formula (I) or a pharmaceutically acceptable salt thereof), and a TMPRSS2 inhibitor.

7. The medicament according to claim 1, wherein (B) is at least one antibody or compound or a pharmaceutically acceptable salt thereof selected from (i) or (ii):
(i) Casirivimab, Imdevimab, Sotrovimab, Tixagevimab, Cilgavimab, or Bebtelovimab,
(ii) Molnupiravir, Remdesivir, AT-527, PF-07321332, PF-00835231, or Camostat, or a pharmaceutically acceptable salt thereof.

8. The medicament according to claim 1, wherein (B) is Casirivimab and Imdevimab.

9. The medicament according to claim 1, wherein (A) and (B) are administered concomitantly.

10. The medicament according to claim 1, which is a combination drug.

11. The medicament according to claim 1, which is used for treating and/or preventing coronavirus disease 2019.

12. The medicament according to claim 1, which is used for treating and/or preventing infective disease due to SARS-CoV-2.

13. An enhancer for (B) according to claim 1, comprising (A) according to claim 1.

14. An enhancer for (A) according to claim 1, comprising (B) according to claim 1.

15. A medicament for administration in combination with (B) according to claim 1, comprising (A) according to claim 1 as an active ingredient.

16. A medicament for administration in combination with (A) according to claim 1, comprising (B) according to claim 1 as an active ingredient.
